SEQUENCE LISTING (11 sequences)

SEQ ID NO: 1 - DNA, 19 bp, Artificial Sequence
  Synthetic Construct
ctgactcttatacacaagt

SEQ ID NO: 2 - DNA, 19 bp, Artificial Sequence
  Synthetic Construct
ctgtctcttgatcagatct

SEQ ID NO: 3 - DNA, 19 bp, Artificial Sequence
  Synthetic Construct
ctgtctcttatacacatct

SEQ ID NO: 4 - DNA, 78 bp, Artificial Sequence
  Synthetic Construct
  misc_feature 20, 21, 23, 24, 26, 28, 29, 31, 32, 34, 36, 37, 39, 41,42: n = A,T,C or G
ctgtctcttatacacatctnnannanwnncnnanannynanncaagcatggtcacttgcagatgtgtataagagacag

SEQ ID NO: 5 - DNA, 78 bp, Artificial Sequence
  Synthetic Construct
  misc_feature 37, 38, 40, 42, 43, 45, 47, 48, 50, 51, 53, 55, 56, 58,59: n = A,T,C or G
ctgtctcttatacacatctgcaagtgaccatgcttgnntnrnntntnngnnwntnntnnagatgtgtataagagacag

SEQ ID NO: 6 - DNA, 61 bp, Artificial Sequence
  Synthetic Construct
  misc_feature 20, 21, 23, 24, 26, 28, 29, 31, 32, 34, 36, 37, 39, 41,42: n = A,T,C or G
ctgtctcttatacacatctnnannanwnncnnanannynanncaagcatggtcacttgcag

SEQ ID NO: 7 - DNA, 36 bp, Artificial Sequence
  Synthetic Construct
ctgtctcttatacacatctgcaagtgaccatgcttg

SEQ ID NO: 8 - DNA, 83 bp, Artificial Sequence
  Synthetic Construct
  misc_feature 39, 40, 42, 43, 45, 47, 48, 50, 51, 53, 55, 56, 58, 60,61: n = A,T,C or G
ctgtctcttatacacatctacggtactcagtctggtcannannanwnncnnanannynanntgcagatgtgtataagagacag

SEQ ID NO: 9 - DNA, 38 bp, Artificial Sequence
  Synthetic Construct
tgaccagactgagtaccgtagatgtgtataagagacag

SEQ ID NO: 10 - DNA, 22 bp, Artificial Sequence
  Synthetic Construct
ctgtctcttatacacatctgca

SEQ ID NO: 11 - DNA, 19 bp, Artificial Sequence
  Synthetic Construct
tgaccagactgagtaccgt

(19) United States
(12) Patent Application Publication
(10) Pub. No.: US 2018/0087050 A1
(43) Pub. Date: Mar. 29, 2018

(54) METHODS OF INSERTING MOLECULAR BARCODES

(71) Applicants: Jianbiao ZHENG, Fremont, CA (US); Changping SHI, San Francisco, CA (US)

(72) Inventors: Jianbiao ZHENG, Fremont, CA (US); Changping SHI, San Francisco, CA (US)

(21) Appl. No.: 15/577,193

(22) PCT Filed: May 26, 2016
(86) PCT No.: PCT/US16/34480

§ 371 Date: Nov. 27, 2017

Related U.S. Application Data
(60) Provisional application No. 62/166,776, filed on May 27, 2015.

(51) Int. Cl. C12N 15/10; C12Q 1/68

(52) U.S. Cl. CPC C12N 15/1068; C12Q 1/6874; C12N 15/1082; C12N 15/1065

(57) ABSTRACT

The present invention provides compositions, methods, and kits for inserting a plurality of synthetic transposons each comprising a different nucleic acid sequence (i.e., molecular barcode) in a target nucleic acid of interest to allow extraction of contiguity information in the target nucleic acid. The molecular barcodes are also useful for reducing amplification or sequencing bias and errors, and for guiding accurate sequence assembly of the target nucleic acid from sequencing reads. The compositions, methods, and kits described herein have many applications, including haplotyping, genome assembly, sequencing of repetitive regions, detection of structural variations and copy number variations, chromosomal conformation analysis, and methylation analysis.

### CROSS REFERENCE TO RELATED APPLICATIONS

This application claims priority benefit of U.S. Provisional Patent Application No. 62/166,776 filed on May 27, 2015, the contents of which are incorporated herein by reference in their entirety.

### FIELD OF THE INVENTION

The present invention relates to the field of genomics, in particular, barcoding and analysis of nucleic acids.

### BACKGROUND OF THE INVENTION

Whole genome sequencing has been used in identifying causes for human diseases. However, there are still some gaps present in the human genome sequences that are not resolved well due to high percentage of repetitive sequences or pseudogenes in the genome, combined with short sequencing reads using current next generation sequencing technologies. Also, there are errors or inconsistencies in sequences determined using different sequencing platforms. The current whole genome sequencing methods depend on reference genomes for assembly, but even the reference genomes contain many gaps. Thus, there are some recent efforts to provide more than one reference genomes for each species. Additionally, current sequencing platforms such as Illumina or Ion Torrent provide short reads in the range of tens to a few hundred nucleobases, which do not readily allow haplotyping by sequencing libraries constructed with conventional methods.

In recent years, the third-generation or single-molecule sequencing technologies, including Pacific Bioscience and Oxford Nanopore technologies, have gained some attention. These new sequencing platforms can sequence single molecules up to 10 kb long, but with large sequencing errors of up to 10-15% per base in comparison to Illumina's error rate of about 0.3% per base. However, repeated sequencing coupled with software tools have allowed correction of sequencing errors to provide correct assembly of the single-molecule sequencing reads, though the cost or scale-up of such technologies still represent practical barriers.

One way to provide more complete coverage for the whole genome is to take advantage of the massively parallel short sequencing reads from Illumina or Ion Torrent sequencing platforms, and complement the short sequencing reads with long sequencing reads from platforms such as Pacific BioSciences. However, haplotyping information for nucleic acids of more than 10 kb long still cannot be easily obtained using the combined methods.

One early method of haplotyping and long-range sequencing is the Long Fragment Read (LFR) method proposed by Complete Genomics. The LFR method involves dilution, and amplification of the nucleic acid templates, followed by sequencing (see, Peters B. A. et al, “Accurate whole-genome sequencing and haplotyping from 10 to 20 human cells” Nature 487: 190-195, 2012). A similar method was presented by Illumina (see, Kaper F. et al, “Whole-genome haplotyping by dilution, amplification, and sequencing” Proc. Natl. Acad. Sci. 110: 5552-5557, 2013). Recently, several methods were developed to address the short-read haplotyping problems and to allow long sequencing with Illumina's short sequencing reads. One technology, Moleculo, which was acquired by Illumina, involves the initial steps of shearing high molecular weight DNA to about 10 kb fragments, end-repairing the 10 kb fragments, and ligation of the fragments with common primers. The ligated fragments are then separate and selected to provide 10 kb templates, which are subsequently diluted in a 384-well plate to one template molecule per well. The diluted 10 kb templates are PCR amplified within the wells, fragmented to 600-800 basepairs, ligated to bar codes and mixed with sequencing primers, pooled together and sequenced with short sequencing reads (see, Amini S. et al, “Haplotype-resolved whole-genome sequencing by contiguity-preserving transposition and combinatorial indexing” Nature Genetics 46: 1343-1349, 2014; McCoy R. C. et al, “Illumina TruSeq synthetic long-reads empower de novo assembly and resolve complex, highly-repetitive transposable elements” PLOS One 9: e10668, 2014). One technology developed by 10× Genomics uses a similar strategy. Instead of diluting individual templates into 384-well plates, the 10× Genomics technology involves a fluidic instrument system to partition the template DNA (see, U.S. patent application publication No. 20150376700).

In U.S. Pat. No. 8,829,171, Steemers et al discloses a method of barcoding target nucleic acids that takes advantage of template mutagenesis using transposons having paired code tags. A plurality of artificial transposons are used in this method, in which each transposon has a transposase recognition site on each end and two barcodes separated by a linker in the middle. The method using paired-code tags can be complicated because of the usage of dual barcodes, and fragmentation sites in the linker for downstream processing of the barcoded nucleic acid templates. Also, paired-code transposons are more difficult to design and produce. In US patent application publication No. US20130203605, Shendure J. A. et al describes a transposon having a bubble structure with two different barcodes, one in each of the two strands of the transposon. The bubble-containing transposon can be used to obtain sequence contiguity information. In chromosomal sequencing, since a separate barcode is used for each strand of the same chromosomal DNA, sequence information from the two strands need to be merged. U.S. Pat. No. 9,328,382 also discloses barcoding methods. Levy and Wigler described a theoretical mutagenesis method for target nucleic acids using partial bisulfite treatment to create unique single-base mutagenesis patterns in individual target molecules (see, Levy D. and Wigler M., “Facilitated sequence counting and assembly by template mutagenesis” Proc. Natl. Acad. Sci. E4632-E4637, 2014). Direct target mutagenesis can be used to solve the problem of sequence assembly and haplotyping. However, a better, simpler method is needed to tag target molecules for sequencing and for providing contiguity information.

Transposases can be used to introduce mutations or insert sequences in nucleic acids. Previously, transposases were used for in vitro or in vivo mutagenesis (e.g., Reznikoff W. S. et al, “Methods for making insertional mutations using a Tn5 synaptic complex”, U.S. Pat. No. 6,159,736) or for producing protein tags (Jarvik J. W., “Methods for producing tagged gene's transcripts and proteins” U.S. Pat. No. 5,652,128). Transposases have also been used to fragment target DNA and to introduce primer binding sequences at the same time (for example, Nextera DNA Sample Prep kit by Illumina/Epicentre).

Molecular barcodes (mBCs) or molecular tags (mTags) have been used in library construction methods to reduce errors introduced by PCR or ligation steps (see, e.g., Kinde I et al, “Detection and quantification of rare mutations with massively parallel sequencing” Proc. Natl. Acad. Sci. USA 108: 9530-9535, 2011; Schmitt M W et al, “Detection of ultra-rare mutations by next-generation sequencing” Proc. Natl. Acad. Sci. USA 109: 14508-14513, 2012). In these cases, introduction of mBCs is typically done after fragmentation. Thus, the mBCs cannot be used to provide sequence contiguity information, which is required for haplotyping or resolving repetitive sequences based on short-read sequencing results.

The disclosures of all publications, patents, patent applications and published patent applications referred to herein are hereby incorporated herein by reference in their entirety.

### BRIEF SUMMARY OF THE INVENTION

The present invention provides compositions, methods, and kits for integration of a plurality of different nucleic acid sequences called molecular barcode or tags in target nucleic acids, which can be used to prepare libraries of template nucleic acids for sequencing.

One aspect of the present application provides a composition comprising a plurality of synthetic transposons, each synthetic transposon comprising a first transposase recognition site, a second transposase recognition site, and a molecular barcode disposed between the first transposase recognition site and the second transposase recognition site, wherein each synthetic transposon comprises a different molecular barcode. In some embodiments, the molecular barcode is double-stranded. In some embodiments, the molecular barcode comprises a single-stranded region. In some embodiments, the molecular barcode is single-stranded.

In some embodiments according to any one of the compositions described herein, each synthetic transposon comprises a terminal hairpin structure. In some embodiments, each synthetic transposon comprises two terminal hairpin structures.

In some embodiments according to any one of the compositions described herein, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated. In some embodiments, wherein the molecular barcode comprises a single-stranded region, the 5′ terminus adjacent to the single-stranded region is phosphorylated.

One aspect of the present application provides a composition comprising a plurality of synthetic transposons, each synthetic transposon comprising a first transposase recognition site, a second transposase recognition site, and a molecular barcode disposed between the first transposase recognition site and the second transposase recognition site, wherein each synthetic transposon comprises a different molecular barcode, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated. Such compositions are referred herein as “strand displacement compatible compositions” or “SDC compositions.”

In some embodiments according to any one of the compositions described above, the first transposase recognition site is different from the second transposase recognition site. In some embodiments, the first transposase recognition site is the same as the second transposase recognition site. In some embodiments, the first transposase recognition site and the second transposase recognition site each comprise a mosaic element (ME).

In some embodiments according to any one of the compositions described above, the molecular barcode comprises at least about 5 (such as at least about any one of 10, 15, 20, or 25) randomly and/or degenerately designed nucleotides.

In some embodiments according to any one of the compositions described above, each synthetic transposon is a DNA transposon or an RNA transposon. In some embodiments, each synthetic transposon comprises a modified nucleotide (such as 5-methyl dC, or LNA).

One aspect of the present application provides a barcoded target nucleic acid comprising a plurality of synthetic transposons inserted randomly or substantially randomly among the endogenous sequence of the barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode. In some embodiments, the molecular barcode is double-stranded. In some embodiments, the molecular barcode comprises a single-stranded region. In some embodiments, the molecular barcode is single-stranded. In some embodiments, each synthetic transposon is flanked by a pair of duplicated sequences endogenous to the barcoded target nucleic acid.

Further provided is a cell comprising any one of the barcoded target nucleic acids described above.

One aspect of the present application provides a method of preparing a library of template nucleic acids, comprising: (a) contacting a target nucleic acid with any one of the compositions described above and a transposase under a condition that allows insertion of at least a portion of the plurality of synthetic transposons into the target nucleic acid to provide a barcoded target nucleic acid; (b) contacting the barcoded target nucleic acid with a polymerase without strand displacement activity, nucleotides, and a ligase to provide a repaired barcoded target nucleic acid; (c) amplifying the repaired barcoded target nucleic acid to provide a plurality of amplified barcoded target nucleic acids; and (d) fragmenting the plurality of amplified barcoded target nucleic acids thereby providing the library of template nucleic acids. In some embodiments, the polymerase without strand displacement activity is T4 DNA polymerase. Such methods are also referred herein as “non-strand displacement methods.”

One aspect of the present application provides a method of preparing a library of template nucleic acids, comprising: (a) contacting a target nucleic acid with any one of the compositions described above and a transposase under a condition that allows insertion of at least a portion of the plurality of synthetic transposons into the target nucleic acid to provide a barcoded target nucleic acid; (b) contacting the barcoded target nucleic acid with a polymerase with strand displacement activity and nucleotides to provide fragments of the repaired barcoded target nucleic acid, wherein each fragment comprises a synthetic transposon at one end; and (c) amplifying the fragments to provide the library of template nucleic acids. In some embodiments, the polymerase with strand displacement activity is a Klenow fragment without 3′-5′ exonuclease activity. In some embodiments, each synthetic transposon comprises a double-stranded molecular barcode. Such methods are also referred herein as “strand displacement methods.”

One aspect of the present application provides a method of preparing a library of template nucleic acids, comprising: (a) contacting a target nucleic acid with any one of the SDC compositions described above, and a transposase under a condition that allows insertion of at least a portion of the plurality of synthetic transposons into the target nucleic acid to provide a barcoded target nucleic acid; (b) contacting the barcoded target nucleic acid with a polymerase without strand displacement activity, nucleotides, and a ligase to provide a repaired barcoded target nucleic acid; (c) contacting the repaired barcoded target nucleic acid with a polymerase with strand displacement activity and nucleotides to provide fragments of the repaired barcoded target nucleic acid, wherein each fragment comprises a synthetic transposon at one end; and (d) amplifying the fragments to provide the library of template nucleic acids. In some embodiments, the polymerase without strand displacement activity is T4 DNA polymerase. In some embodiments, the polymerase with strand displacement activity is a Klenow fragment without 3′-5′ exonuclease activity. In some embodiments, the method further comprises amplifying (such as by PCR) the template nucleic acids. Such methods are also referred herein as “combination methods.”

In some embodiments according to any one of the methods of preparing a library of template nucleic acids described above, the target nucleic acid is contacted with the plurality of synthetic transposons and the transposase in vitro. In some embodiments, the plurality of synthetic transposons and the transposase are pre-mixed prior to contacting the target nucleic acid. In some embodiments, the target nucleic acid is contacted with the plurality of synthetic transposons and the transposase in vivo.

In some embodiments according to any one of the methods of preparing a library of template nucleic acids described above, the transposase is Tn5 transposase.

In some embodiments according to any one of the methods of preparing a library of template nucleic acids described above, the target nucleic acid is selected from the group consisting of cDNA, genomic DNA, bisulfite-treated DNA, and crosslinked DNA.

In some embodiments according to any one of the methods of preparing a library of template nucleic acids described above, the plurality of synthetic transposons are inserted into the target nucleic acid at a frequency of at least once per about 500 bases (such as at least once per about 250 bases, or at least once per about 150 bases).

In some embodiments according to any one of the methods of preparing a library of template nucleic acids described above, the method further comprises diluting the barcoded target nucleic acid into a plurality of compartments.

In some embodiments according to any one of the methods of preparing a library of template nucleic acids described above, the amplifying is PCR amplification. In some embodiments, the amplifying is whole genome amplification. In some embodiments, the amplifying is amplifying of targeted sequences, such as exome.

One aspect of the present application provides a method of analyzing a target nucleic acid, comprising: (a) preparing a library of template nucleic acids from the target nucleic acid using any one of the methods of preparing a library of template nucleic acids described above; (b) sequencing the library of template nucleic acids to obtain sequencing reads; and (c) assembling a contiguous sequence of the target nucleic acid from the sequencing reads based on the molecular barcodes of the synthetic transposons in the template nucleic acids. In some embodiments, the sequencing is massively parallel shotgun sequencing. In some embodiments, step (c) comprises: (i) identifying sequences of the synthetic transposons in the sequencing reads; (ii) aligning sequencing reads having the same molecular barcodes in the synthetic transposons to provide aligned sequencing reads; (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence. In some embodiments, the method is used for genome assembly, haplotyping, detection of mutation (such as substitution, indel, structural variation, or copy number variation), chromosomal conformation analysis, or methylation analysis.

Further provided are kits and articles of manufacture useful for any of the methods described above.

One aspect of the present application provides a kit for preparing a library of template nucleic acids, comprising: (a) any one of the compositions (including SDC compositions) described above; (b) a transposase that recognizes the first transposon recognition site and the second transposon recognition site; and (c) instructions for preparing a library of template nucleic acids. In some embodiments, the molecular barcode is double-stranded. In some embodiments, the molecular barcode comprises a single-stranded region. In some embodiments, the molecular barcode is single-stranded. In some embodiments, the kit further comprises a polymerase without strand displacement activity (such as T4 DNA polymerase). In some embodiments, the kit further comprises a polymerase with strand displacement activity (such as a Klenow fragment without 3′-5′ exonuclease activity). In some embodiments, the kit further comprises a ligase. In some embodiments, the transposase is Tn5 transposase (such as Tn5 transposase with enhanced activity, for example, EZ-Tn5™).

It is understood that aspects and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1A depicts an exemplary synthetic transposon comprising a molecular barcode sequence (101; mBC) flanked by a pair of transposase recognition sites on each side (102 and 103).

FIG. 1B depicts an exemplary synthetic transposon comprising a molecular barcode sequence (101; mBC) flanked by a pair of transposase recognition sites on each side (102 and 103), and additional sequences (104 and 105) outside the transposase recognition sites. The additional sequences can be removed during the insertion of sequence comprising 102, 101, and 103 in a target nucleic acid. The 5′ ends of the strands may or may not be phosphorylated depending on the needs.

FIG. 1C depicts an exemplary synthetic transposon comprising a single-stranded molecular barcode region (101; mBC) disposed between two transposase recognition sites (102, 103). The 5′ ends of the strands may or may not have phosphate groups depending on the needs.

FIG. 1D depicts an exemplary synthetic transposon comprising a molecular barcode sequence (101; mBC) flanked by a pair of transposase recognition sites on each side (102 and 103), additional sequences (104 and 105) flanking the transposase recognition sites, and terminal hairpin structures on both ends.

FIG. 1E depicts an exemplary synthetic transposon comprising a single-stranded molecular barcode sequence (101; mBC) disposed between two transposase recognition sites (102, 103), and terminal hairpin structures on both ends.

FIG. 1F depicts an exemplary synthetic transposon comprising a molecular barcode sequence (101; mBC) flanked by a pair of transposase recognition sites on each side (102 and 103), an additional sequence (105) flanking one transposase recognition site (103), and a terminal hairpin structure flanking the additional sequence (105) on one end.

FIG. 1G depicts an exemplary synthetic transposon comprising a molecular barcode sequence (101; mBC) flanked by a pair of transposase recognition sites on each side (102 and 103), additional sequences (104 and 105) flanking the transposase recognition sites, and a terminal hairpin structure flanking one additional sequence (105) on one end.

FIG. 1H depicts an exemplary synthetic transposon comprising a single-stranded molecular barcode region (101; mBC) disposed between two transposase recognition sites (102, 103), in which the 5′ terminal nucleotide of the continuous strand (102+101+103) and the 5′ terminal nucleotide of the bottom (i.e. noncoding or complementary) strand of 103 have free 5′ hydroxyl groups, and the 5′ terminal nucleotide of the top (i.e., coding) strand of 102 has a 5′ phosphate group.

FIG. 2A depicts an exemplary double-stranded synthetic transposon (top) and an exemplary method for preparing the double-stranded synthetic transposon (bottom). The synthetic transposon has a 19-bp mosaic Tn5 recognition sequence (201 and 202) on each end of a double-stranded molecular barcode region (203) including 15 randomly designed nucleotides dispersed among 25 degenerately designed and fixed bases. The fixed bases in the molecular barcode region facilitate formation of dimers of transposase molecules bound to the transposase recognition sites. Additionally, the fixed bases allow easy preparation of the double-stranded synthetic transposon from two oligos (204 and 205) that hybridize via the fixed bases, while minimizing the impact of self-hairpin structures if the two transposase recognition sites are inverse repeats. Unused single-stranded DNA can be removed by Exonuclease I or purified away from the desired double-stranded synthetic transposons. In some embodiments, the two transposase recognition sites have different sequences to allow easy preparation of the synthetic transposons and to minimize issues in downstream applications. Symbols used in the figure are as follows: n=any base of A/C/G/T; B=C/G/T; D=A/G/T; H=A/C/T; V=A/C/G; W=A/T; S=C/G; R=A/G; Y=C/T. The nucleotides can be deoxyribonucleotides or ribonucleotides.

FIG. 2B depicts an exemplary synthetic transposon comprising a 19-bp mosaic Tn5 transposase recognition sequence (201b and 202b) on each end, and a partially single-stranded molecular barcode (203b) with 15 randomly designed nucleotides (Ns) mixed with degenerately designed and fixed nucleotides having the same 5′ terminal groups as in the synthetic transposon of FIG. 1H. n=any base of A/C/G/T; B=C/G/T; D=A/G/T; H=A/C/T; V=A/C/G; W=A/T; S=C/G; R=A/G; Y=C/T. The nucleotides can be deoxyribonucleotides or ribonucleotides.

FIG. 3 depicts transposition of a double-stranded genomic DNA inserted with a plurality of synthetic transposons catalyzed by Tn5 transposase. For clarity purposes, a single insertion site is illustrated. Tn5 binds the mosaic elements (ME1, ME2, or 302, 303) of the synthetic transposon and forms a dimeric complex. Random transposition of the Tn5/synthetic transposon complex into target DNA leads to a 9-nucleotide (i.e., 9-nt) single-stranded gap on each side of each inserted synthetic transposon. Each synthetic transposon can have a different mBC sequence (301) by incorporating about 20 randomly designed nucleotides (or 10¹² possibilities) in the mBC. For example, the synthetic transposons having different mBCs can be inserted into 2×10⁷ sites in each human genome at an average distance of about 150-bp, to provide barcoded genomic DNA molecules each having a different barcoding pattern and barcoding sequences.

FIG. 4 depicts an exemplary method of preparing a library of template nucleic acids comprising steps (a)-(d). Step (a) starts with the exemplary genomic DNA inserted with a plurality of synthetic transposons as shown in FIG. 3. In step (b), a DNA polymerase with strand displacement activity is used to fill in the 9-nt single-stranded gap generated by the Tn5 transposition events. In step (c), the strand displacement activity of the DNA polymerase displaces one strand of the inserted synthetic transposon until separation of the extended strands from the original synthetic transposon strands and completion of the gap filling in (d). The method results in fragments of the barcoded genomic DNA. Both ends of each fragment are characterized by a different synthetic transposon sequence followed by a duplicated 9-nt endogenous gap sequence, thereby providing contiguity information among the fragments.

FIG. 5 depicts another exemplary method of preparing a library of template nucleic acids having inserted synthetic transposons for maintaining contiguity information. In step (a), a plurality of synthetic transposons is inserted into a target DNA using Tn5 transposase without breaking the DNA. The modified DNA is repaired by incubation with a DNA polymerase without strand displacement activity and dNTPs to fill-in the 9-nt single-stranded gaps, and with a ligase for nick sealing (step (b)). The resulting DNA is amplified by multiple displacement amplification (MDA) or other amplification methods in (c), followed by fragmentation (d) to provide a library of template nucleic acids, which is subject to end repair, adaptor ligation, and optional amplification steps to construct a library for sequencing (step (e)).

FIG. 6 depicts an exemplary method for library construction from short double-stranded (ds) DNA fragments (601) such as fragments produced in step (d) of FIG. 5. The dsDNA fragments are end repaired to form fragments with blunt ends (602), subjected to dA addition (603), ligation to adaptors (604) to form the product (605) that allows amplification with common primers and addition of sample tags (606).

FIG. 7 depicts an exemplary method for correcting errors or bias (marked as “X”) by using molecular barcodes of the Tn5 synthetic transposons (ST) found in the sequencing reads for alignment and clustering of the sequencing reads to generate a consensus sequence of a single template molecule. The different molecular barcodes and the 9-nt duplicate gap sequences on each side of the synthetic transposon serve as identifiers to cluster the sequencing reads having the same barcodes and 9-nt sequences. Clustering allows correction of amplification or sequencing errors, and elimination of amplification bias. Individual sequencing reads are then assembled together to obtain a phased uninterrupted sequence.

FIG. 8 depicts an exemplary method for correcting errors (marked as “X”) using molecular barcodes of the Tn5 synthetic transposons (ST) found in the sequencing reads for alignment and clustering of the sequencing reads to generate a consensus sequence of a single template molecule. The transposase recognition sites flanking the molecular barcodes serve as identifiers to pin point the location of the molecular barcodes, which can be indexed and aligned to the next fragment having identical 9-nt sequences and molecular barcode sequences.

### DETAILED DESCRIPTION OF THE INVENTION

The present application discloses compositions, methods and kits for inserting a plurality of different molecular barcodes carried by synthetic transposons into target nucleic acids, which are useful for scalable and precise assembly and quantitation of the target nucleic acid molecules based on next-generation sequencing reads of libraries constructed from the barcoded target nucleic acids. The methods use integrases or transposases to insert synthetic transposons carrying different molecular barcodes randomly or substantially randomly into the target nucleic acids at distances from about tens of bases to about tens of kilobases or more, thereby preserving the contiguity information in the target nucleic acid during later steps of sequencing library preparation. As each inserted molecular barcode sequence is different, sequencing reads with identical molecular barcodes are derived from a single original target molecule. In some cases, duplicated endogenous sequences of the target nucleic acid flanking the synthetic transposons provide further contiguity information that can be used in combination with the molecular barcodes to trace the sequencing reads back to original target molecules. Thus, by deriving consensus sequences from clustered sequencing reads having the same molecular barcodes, amplification or sequencing errors introduced later in the library construction or sequencing process can be corrected. Additionally, amplification bias can be eliminated by counting all sequencing reads mapping to the same target nucleic acid as a single molecule. In this way, the targeted nucleic acid molecules can be quantified accurately, and assembled with high precision. The compositions, methods, kits and analysis software described herein are therefore very useful for many applications, including haplotyping, de novo assembly of whole genomes or long contiguous sequences, sequencing of repetitive regions, detection of structural variations and copy number variations, methylation analysis and many others.

The compositions and methods of the present application differ from currently known molecular barcoding methods for extracting contiguity information in many ways. For example, synthetic transposons having a single-stranded or partially single-stranded molecular barcode are disclosed herein. The single-stranded region can provide higher structural flexibility and facilitate transposase dimer formation, thereby improving the efficacy of insertion of the synthetic transposons in the target nucleic acids. High efficacy of insertion is particularly desirable in embodiments of methods that require high frequency and/or low sequence bias in the transposition events into a long, contiguous target nucleic acid, such as an intact chromosome. Methods of synthetic transposon insertion described in the present application can be applied in vitro, or in vivo, both of which are compatible with a variety of downstream sequencing library construction workflows. The in vivo methods can be particularly desirable for applications that rely heavily on contiguity information of genomic DNA, including, for example, haplotyping and detection of chromosomal structural and copy number variations. Additionally, in some embodiments, by using a polymerase with strand displacement activity following insertion of the synthetic transposons, fragments of target nucleic acids having barcoded ends are produced. Sequencing reads from such fragments are easy to cluster and analyze.

Accordingly, in one aspect, the present application provides a composition comprising a plurality of synthetic transposons, each comprising a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region. In some embodiments, the molecular barcode is single-stranded.

In one aspect, the present application provides a method of preparing a library of template nucleic acids, comprising: (a) contacting a target nucleic acid with a composition comprising a plurality of synthetic transposons and a transposase under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region; (b) contacting the barcoded target nucleic acid with a polymerase without strand displacement activity, nucleotides, and a ligase to provide a repaired barcoded target nucleic acid; (c) amplifying the repaired barcoded target nucleic acid to provide a plurality of amplified barcoded target nucleic acids; and (d) fragmenting the plurality of amplified barcoded target nucleic acids thereby providing the library of template nucleic acids. In some embodiments, the molecular barcode is single-stranded.

In one aspect of the present application, there is provided a method of preparing a library of template nucleic acids, comprising: (a) contacting a target nucleic acid with a composition comprising a plurality of synthetic transposons, and a transposase under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand displacement activity, nucleotides, and a ligase to provide a repaired barcoded target nucleic acid; (c) contacting the repaired barcoded target nucleic acid with a polymerase with strand displacement activity and nucleotides to provide fragments of the repaired barcoded target nucleic acid, wherein each fragment comprises a synthetic transposon at one end; and (d) amplifying the fragments to provide the library of template nucleic acids.

In another aspect of the present application, there is provided a method of preparing a library of template nucleic acids, comprising: (a) contacting a target nucleic acid with a composition comprising a plurality of synthetic transposons and a transposase under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity and nucleotides to provide fragments of the repaired barcoded target nucleic acid, wherein each fragment comprises a synthetic transposon at one end; and (c) amplifying the fragments to provide the library of template nucleic acids.

### Compositions

One aspect of the present application provides a composition comprising a plurality of synthetic transposons each comprising a first transposase recognition site, a second transposase recognition site and a molecular barcode disposed between the first transposase recognition site and the second transposase recognition site, wherein each synthetic transposon comprises a different molecular barcode. The plurality of synthetic transposons is also referred herein as “Random synthetic transposons,” “STs,” or “RSTs.” The molecular barcode comprises a plurality of nucleotides that are randomly or degenerately designed, thereby yielding a highly diverse sequence that can be used to identify each individual synthetic transposon, and the target nucleic acid or fragment thereof that the synthetic transposon inserts into.

In some embodiments, there is provided a composition comprising a plurality of synthetic transposons, each synthetic transposon comprising a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region. In some embodiments, the molecular barcode is single-stranded. In some embodiments, the first transposase recognition site is different from the second transposase recognition site. In some embodiments, the first transposase recognition site is the same as the second transposase recognition site. In some embodiments, the first transposase recognition site and/or the second transposase recognition site each comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA). In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated.

In some embodiments, there is provided a composition comprising a plurality of complexes each comprising a synthetic transposon and a transposase, wherein each synthetic transposon comprises a first transposase recognition site, a second transposase recognition site and a molecular barcode disposed between the first transposase recognition site and the second transposase recognition site, wherein each synthetic transposon comprises a different molecular barcode, wherein the molecular barcode comprises a single-stranded region, and wherein the transposase is bound to the first transposase recognition site and the second transposase recognition site. In some embodiments, the transposase is a dimeric transposase. In some embodiments, the transposase is Tn5 transposase, such as a hyperactive Tn5 transposase, for example, EZ-Tn5™. In some embodiments, the first transposase recognition site is different from the second transposase recognition site. In some embodiments, the first transposase recognition site is the same as the second transposase recognition site. In some embodiments, the first transposase recognition site and/or the second transposase recognition site each comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA). In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated.

In some embodiments, there is provided a composition comprising a plurality of synthetic transposons, each synthetic transposon comprising a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated. In some embodiments, the first transposase recognition site is different from the second transposase recognition site. In some embodiments, the first transposase recognition site is the same as the second transposase recognition site. In some embodiments, the first transposase recognition site and/or the second transposase recognition site each comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA).

In some embodiments, there is provided a composition comprising a plurality of complexes each comprising a synthetic transposon and a transposase, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated. In some embodiments, the transposase is a dimeric transposase. In some embodiments, the transposase is Tn5 transposase, such as a hyperactive Tn5 transposase, for example, EZ-Tn5™. In some embodiments, the first transposase recognition site is different from the second transposase recognition site. In some embodiments, the first transposase recognition site is the same as the second transposase recognition site. In some embodiments, the first transposase recognition site and/or the second transposase recognition site each comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA).

In some embodiments, there is provided a composition comprising a plurality of synthetic transposons, each synthetic transposon comprising a first transposase recognition site, a second transposase recognition site, and a double-stranded molecular barcode disposed between the first transposase recognition site and the second transposase recognition site, and wherein each synthetic transposon comprises a different molecular barcode. In some embodiments, the first transposase recognition site is different from the second transposase recognition site. In some embodiments, the first transposase recognition site is the same as the second transposase recognition site. In some embodiments, the first transposase recognition site and/or the second transposase recognition site comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA). In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated.

In some embodiments, there is provided a composition comprising a plurality of complexes each comprising a synthetic transposon and a transposase, wherein each synthetic transposon comprises a first transposase recognition site, a second transposase recognition site, and a double-stranded molecular barcode disposed between the first transposase recognition site and the second transposase recognition site, wherein each synthetic transposon comprises a different molecular barcode, and wherein the transposase is bound to the first transposase recognition site and the second transposase recognition site. In some embodiments, the transposase is a dimeric transposase. In some embodiments, the transposase is Tn5 transposase, such as a hyperactive Tn5 transposase, for example, EZ-Tn5™. In some embodiments, the first transposase recognition site is different from the second transposase recognition site. In some embodiments, the first transposase recognition site is the same as the second transposase recognition site. In some embodiments, the first transposase recognition site and/or the second transposase recognition site comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA). In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated.

In some embodiments, there is provided a composition comprising random synthetic transposons (RSTs), each comprising: (a) a first nucleic acid transposase recognition sequence, (b) a second nucleic acid transposase recognition sequence; and (c) a plurality of unique and fixed bases called molecular barcode or tag between the first and second transposase recognition sequences. In some embodiments, the first transposase recognition sequence could be the same as the second transposase recognition sequence or different from second transposase recognition sequence to minimize downstream complication due to intramolecular hairpin formation of the transposase recognition sequences. In some embodiments, at least one of the transposase recognition sequences is a mosaic element (ME). In some embodiments, the transposase that bind the RSTs is Tn5. In some embodiments, either of the transposase recognition sequences may have 5′ phosphate group if no additional sequences are outside. In some embodiments, additional sequences outside the transposase recognition sequences could be optionally added. In some embodiments, the molecular barcode region is a single stranded nucleic acid sequence. In some embodiments, some or all of the nucleotides in the synthetic random transposon are deoxyribonucleotides, ribonucleotides or modified bases (i.e., nucleotides).

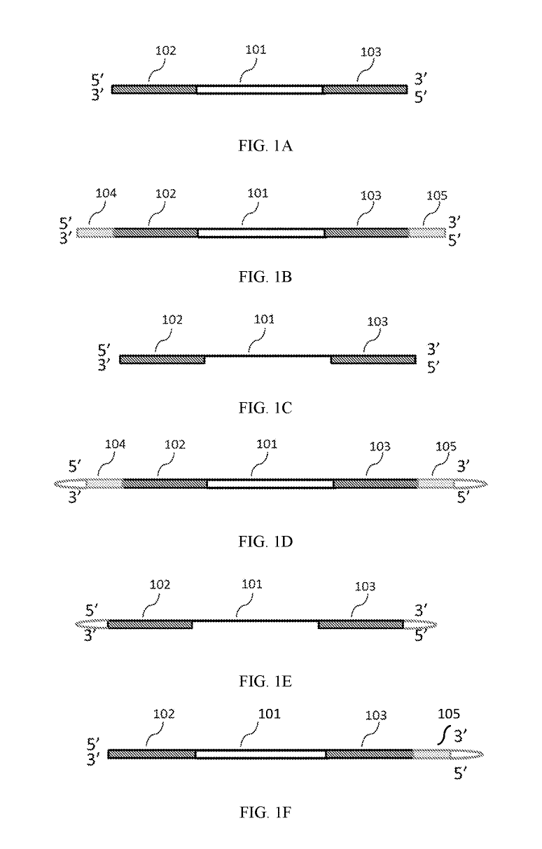

Exemplary synthetic transposons are shown in FIGS. 1A-1H. In some embodiments, there is provided a composition comprising a plurality of random synthetic transposons (RSTs) each consisting of a molecular barcode comprising a plurality of randomly or degenerately designed nucleotides (which could be mixed with fixed bases between Ns, e.g., sequence 101 of FIG. 1A) flanked by a pair of transposase recognition sites on each side (sequences 102 and 103 of FIG. 1A). In some embodiments, the plurality of randomly or degenerately designed nucleotides consists of about 10-30 nucleotides. This design can be varied in many ways. For example, in some embodiments, there is provided a composition comprising a plurality of random synthetic transposons each consisting of two extra sequences (e.g., 104 and 105 of FIG. 1B), two transposase recognition sites, and a molecular barcode comprising a plurality of randomly or degenerately designed nucleotides, wherein each of the extra sequences flanks the outside of one transposase recognition site, wherein the two transposase recognition sites flank the molecular barcode, and wherein the extra sequences are removed during transposition events (see, for example, FIG. 1B). In some embodiments, there is provided a composition comprising a plurality of synthetic transposons, each synthetic transposon comprising a first transposase recognition site and a second transposase recognition site flanking a single-stranded molecular barcode comprising a plurality of randomly or degenerately designed nucleotides (see, for example, FIG. 1C). In some embodiments, one or both ends of the synthetic transposon comprise a terminal hairpin structure. For example, the double-stranded synthetic transposons in FIG. 1B may be modified with terminal hairpin structures on both ends (e.g., FIG. 1D), or a terminal hairpin structure on one end only (e.g., FIG. 1G). Synthetic transposons with single-stranded molecular barcodes as shown in FIG. 1C may also be modified with hairpin structures on both ends (e.g., FIG. 1E). Double-stranded synthetic transposons in FIG. 1A may be modified by including one additional sequence and a terminal hairpin structure on one end only (e.g., FIG. 1F). The randomly or degenerately designed molecular barcode of the sequence 101 in all exemplary synthetic transposons discussed herein can be used to identify the lineage of molecular amplification. Therefore, any further replication from the original target molecule into which the synthetic transposons insert into can be clustered back to the original target molecule. The additional sequences (e.g., 104 and 105) may be used to provide sequences for primer hybridization, which allows convenient amplification of precursor oligonucleotides to prepare the synthetic transposons. The synthetic transposons may adopt other formats not illustrated in FIGS. 1A-1H. For example, the molecular barcode can be partially single-stranded.

The composition may comprise any number of synthetic transposons having different molecular barcodes. In some embodiments, the composition comprises a single copy of each synthetic transposon having a different molecular barcode. In some embodiments, the composition comprises more than one copy of each synthetic transposon having a different molecular barcode. In some embodiments, the plurality of synthetic transposons have at least about any one of 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, or more different molecular barcodes. In some embodiments, the plurality of synthetic transposons have at least about any one of 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, or more sources of clonal molecular barcodes.

The molecular barcode of each synthetic transposon is different because it contains nucleotide sequences comprising randomly designed (i.e., having any of the four nucleobases A, C, T, G) or degenerately designed (i.e., having one of a set of at least two types of nucleobases, for example, B=C/G/T; D=A/G/T; H=A/C/T; V=A/C/G; W=A/T; S=C/G; R=A/G; Y=C/T) nucleotides. The nucleotide can be a ribonucleotide, or a deoxyribonucleotide. The molecular barcode can thus be used to identify a particular fragment of a target nucleic acid that the synthetic transposon carrying the molecular barcode inserts into. The molecular barcode may further comprise nucleotides having the same identity for all synthetic transposons (i.e. “fixed” or specifically designed nucleotides). The additional fixed nucleotides or sequences can be placed on either side of the randomly or degenerately designed sequence or interspersed among the randomly or degenerately designed nucleotides.

In some embodiments, the molecular barcode comprises double-stranded regions. In some embodiments, the molecular barcode is double-stranded. In some embodiments, the molecular barcode is single-stranded. In some embodiments, the molecular barcode is partially single-stranded (i.e., partially double-stranded). In some embodiments, the molecular barcode has a single-stranded region having at least about any one of 1, 2, 3, 4, 5, 6, 7, 8, 9, 10, 11, 12, 13, 14, 15, 16, 17, 18, 19, 20, 25, 30, 35, 40, 50 or more nucleotides. In some embodiments, the randomly and/or degenerately designed nucleotides in the molecular barcode are in single-stranded region of the molecular barcode. In some embodiments, the double-stranded region of the at least partially single-stranded molecular barcode comprises fixed nucleotides. In some embodiments, the double-stranded region of the at least partially single-stranded molecular barcode consists essentially of fixed nucleotides. In some embodiments, the synthetic transposon further comprises fixed nucleotides outside the molecular barcode and between the first transposase recognition site and the second transposase recognition site. Continuous sequences consisting of fixed nucleotides (such as “stuff sequences”) as part of the molecular barcode or outside the molecular barcode may facilitate preparation of the synthetic transposon, library preparation steps (such as by providing sites for primers to hybridize to), and/or data analysis steps (such as for easy alignment and clustering of sequencing reads).

In some embodiments, the molecular barcode comprises at least about any one of 5, 10, 15, 20, 25, 30, 35, 40, 45, 50, 60, 70 80, 90, 100 or more consecutive nucleotides. In some embodiments, the molecular barcode comprises at least about any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40 or more randomly designed nucleotides. In some embodiments, the molecular barcode comprises at least about any one of 1, 2, 3, 4, 5, 6, 7, 8, 9, 10, 11, 12, 13, 14, 15, 16, 17, 18, 19, 20, 25, 30, 35, 40 or more degenerately designed nucleotides. In some embodiments, the molecular barcode comprises at least about any one of 1, 2, 3, 4, 5, 6, 7, 8, 9, 10, 11, 12, 13, 14, 15, 16, 17, 18, 19, 20, 25, 30, 35, 40 or more fixed (i.e., specifically designed) nucleotides. In some embodiments, the molecular barcode is a mixture of randomly designed, degenerately designed or fixed nucleotides. The number of randomly and/or degenerately designed nucleotides in the molecular barcode depends on the actual need. For example, a long target nucleic acid (such as chromosome) may need a plurality of synthetic transposons with higher diversity, i.e., a large number of randomly and/or degenerately designed nucleotides, to provide enough distinct molecular barcodes to tag the large number of segments of the target nucleic acid in order to extract contiguity information. By contrast, a short target nucleic acid, such as a plasmid of a few kilobases long, may only need a small number of randomly and/or degenerately designed nucleotides to provide enough distinct molecular barcodes for tagging. In some cases, duplicated sequences endogenous to the target nucleic acid flanking the insertion sites of the synthetic transposons (e.g., 9-nt duplicate sequences for Tn5 transposase) may be used in combination with the molecular barcodes in the synthetic transposons to provide contiguity information for the target nucleic acids. Having both randomly designed and specific nucleotides may minimize potential undesired non-specific interactions during the process of synthesizing the synthetic transposons. In some embodiments, the synthetic transposon comprises nucleotides flanking the transposase recognition sites at one or both ends of the synthetic transposon.

In some embodiments, the molecular barcode has one, two, or three polynucleotide strands. The polynucleotide strands have consecutive nucleotides linked in a 5′ to 3′ fashion. In some embodiments, different polynucleotide strands may hybridize to each other to form double-stranded regions. In some embodiments, regions within a polynucleotide strand may be complementary to each other and hybridize to form hairpin structures. In some embodiments, the molecular barcode comprises two polynucleotide strands that are complementary to each other. In some embodiments, the molecular barcode comprises a continuous polynucleotide strand, and a discontinuous strand comprising two polynucleotide strands, wherein the two discontinuous strands hybridize to the continuous polynucleotide strand. In some embodiments, the molecular barcode comprises a terminal hairpin structure at one end. In some embodiments, the molecular barcode comprises a first terminal hairpin structure at a first end, and a second terminal hairpin structure at the second end. In some embodiments, the molecular barcode has one double-stranded end. In some embodiments, the molecular barcode has two double-stranded ends.

A transposase recognition site can include two complementary nucleic acid sequences, e.g., a double-stranded nucleic acid or a hairpin nucleic acid, that comprise a substrate for a transposase or integrase. The length of the transposase recognition sites in natural transposons recognized by a transposase could vary depending on the nature of the transposase, including about 4-bp for Ty1 transposons, about 19-bp for Tn5 transposons, about 51-bp for Mu transposons, about 90-bp on the right side end of Tn7 transposons (Tn7R) and about 165-bp on the left side end of Tn7 transposons (Tn7L). The synthetic transposons described herein have transposase recognition sites with sequences and lengths recognizable by a natural or modified (such as hyperactive) transposase or integrase.

The transposase or integrase may bind to the transposase recognition site and insert the transposase recognition site into a target nucleic acid. In nature, transposase is an enzyme that binds to both ends (i.e., transposase recognition sites) of a transposon and catalyzes the movement of the transposon from one part of the genome to another part of the genome by a cut and paste mechanism or a replicative transposon mechanism. As used herein, “transposition,” “insertion,” and “integration” are used interchangeably to refer to the movement of a synthetic or natural transposon into a target nucleic acid. The compositions, methods and kits described herein may use transposase-recognized synthetic transposons, or integrase-recognized synthetic transposons.

Also provided herein are compositions comprising a plurality of complexes each comprising a transposase bound to the transposase recognition sites of any of the synthetic transposons (or random synthetic transposons) described herein. The complexes can be prepared by mixing the plurality of synthetic transposons and the transposase. In some embodiments, the synthetic transposons and the transposase are incubated for at least about any one of 1 minute, 5 minutes, 10 minutes, 30 minutes, 1 hour or more to form the complexes. In such complexes, the transposase can form a functional complex with one or more transposes recognition sites, and is capable of catalyzing a transposition reaction. Some embodiments can include the use of a hyperactive Tn5 transposase and a Tn5-type transposase recognition site (Goryshin, I. and Reznikoff, W. S., J. Biol. Chem., 273: 7367, 1998), or MuA transposase and a Mu transposase recognition site comprising R1 and R2 end sequences (Mizuuchi, K., Cell, 35: 785, 1983; Savilahti, H, et al., EMBO J., 14: 4893, 1995). The first transposase recognition site and the second transposase recognition site can have the same or different sequences. In some embodiments, the first transposase recognition site is an inverse repeat of the second transposase recognition site. In some embodiments, the first transposase recognition site and the second transposase recognition site have mismatching sequences. For example, Tn5 transposase recognizes two 19-bp transposase recognition sequences named outside end (“OE”, SEQ ID NO:1 CTGACTCTTATACACAAGT) and inside end (“IE”, SEQ ID NO:2 CTGTCTCTTGATCAGATCT), which have different sequences. OE and IE may be used in synthetic transposons of the present application. In some embodiments, the first transposase recognition site and the second transposase recognition site are mosaic ends (also known as “mosaic elements,” or “MEs”), which are hybrid sequences of naturally occurring transposase recognition sites at the ends of a transposon, and the MEs can have higher affinity to the transposase or be hyperactive in transposition events compared to naturally occurring transposase recognition sites. An exemplary mosaic element suitable for use in the synthetic transposons described herein has the sequence CTGTCTCTTATACACATCT (SEQ ID NO:3), which is recognized by a hyperactive Tn5 transposase (e.g., EZ-Tn5™ Transposase, Epicentre Biotechnologies, Madison, Wis., USA). More examples of transposition systems that can be used with certain embodiments provided herein include Staphylococcus aureus Tn552 (Colegio O R et al., J. Bacteriol., 183: 2384-8, 2001; Kirby C et al., Mol. Microbiol., 43: 173-86, 2002), Ty1 (Devine S E, and Boeke J D., Nucleic Acids Res., 22: 3765-72, 1994 and International Patent Application No. WO 95/23875), Transposon Tn7 (Craig, N L, Science. 271: 1512, 1996; Craig, N L, Review in: Curr Top Microbiol Immunol., 204: 27-48, 1996), Tn/O and IS10 (Kleckner N, et al., Curr Top Microbiol Immunol., 204: 49-82, 1996), Mariner transposase (Lampe D J, et al., EMBO J., 15: 5470-9, 1996), Tc1 (Plasterk R H, Curr Top Microbiol Immunol, 204: 125-43, 1996), P Element (Gloor, G B, Methods Mol. Biol., 260: 97-114, 2004), Tn3 (Ichikawa H, and Ohtsubo E., J. Biol. Chem. 265: 18829-32, 1990), bacterial insertion sequences (Ohtsubo, F and Sekine, Y, Curr. Top. Microbiol. Immunol. 204: 1-26, 1996), retroviruses (Brown P O, et al., Proc Natl Acad Sci USA, 86: 2525-9, 1989), and retrotransposon of yeast (Boeke J D and Corces V G, Annu Rev Microbiol. 43: 403-34, 1989), the disclosures of which are incorporated herein by reference in their entireties. Commercial transposases for mutagenesis are available, for example, from NEB, Epicentre (now part of Illumina) and Finnzymes.

Transposases can be multimeric. For example, Tn5 and Mu transposases are homodimers of a single polypeptide (Tnp or MuA respectively), while Tn7 transposase comprises 3 different polypeptides (TsnA/B/C). In order to form a complex, the nucleic acid disposed between the first transposase recognition site and the second transposase recognition site are designed to have a suitable length and structural flexibility to avoid steric hindrance and allow interaction among the transposase monomers bound to the transposase recognition sites. For example, the length of the nucleic acid sequence comprising the molecular barcode can be at least about any one of 5, 10, 15, 20, 25, 30, 35, 40, 45, 50, 60, 70 80, 90, 100 or more nucleotides. In some embodiments, the length of the nucleic acid sequence comprising the molecular barcode is about 40-80 nucleotides. In some embodiments, the nucleic acid disposed between the first transposase recognition site and the second transposase recognition sites comprises a single-stranded region or is single-stranded. Synthetic transposons with single-stranded regions can be bent easily without the use of lengthy sequences between the transposase recognition sites, thereby facilitating binding and insertion of the synthetic transposon by the transposase.

In some embodiments, one or more of the 5′ ends (also referred herein as 5′ termini) of the polynucleotide strands in the synthetic transposons are phosphorylated, or the 5′ terminal nucleotide has a 5′ phosphate group. Phosphorylated 5′ ends facilitate ligation to other nucleic acids, such as adaptors, extended, or gap-filled nucleic acid strands (e.g., for nick-sealing). For example, in some embodiments, wherein each synthetic transposon comprises two double-stranded ends with no terminal hairpin structures, the 5′ termini of the two double-stranded ends are phosphorylated. In some embodiments, wherein the synthetic transposon comprises two continuous polynucleotide strands, the 5′-ends of both continuous polynucleotide strands are phosphorylated. In some embodiments, one or more of the 5′ ends (also referred herein as 5′ termini) of the polynucleotide strands in the synthetic transposons are unphosphorylated, for example, the 5′ terminal nucleotide has a 5′ free hydroxyl group. In some embodiments, wherein each synthetic transposon comprises two double-stranded ends with no terminal hairpin structures, the 5′ termini of the two double-stranded ends are unphosphorylated. In some embodiments, wherein the synthetic transposon comprises two continuous polynucleotide strands, the 5′-ends of both continuous polynucleotide strands are phosphorylated. In some embodiments, wherein the molecular barcode comprises a single-stranded region, the 5′ end adjacent to the single-stranded region, i.e., the 5′ end at the junction of single-stranded and double-stranded regions of the molecular barcode may be phosphorylated or unphosphorylated. In some embodiments, wherein each synthetic transposon comprises two double-stranded ends with no terminal hairpin structures, and wherein the molecular barcode comprises a single-stranded region, the 5′ termini of the two double-stranded ends are unphosphorylated, and the 5′ terminus adjacent to the single-stranded region is phosphorylated, i.e., the 5′ terminal nucleotide adjacent to the single-stranded region has a 5′ phosphate group. Synthetic transposons having 5′ hydroxyl ends may be phosphorylated in the library construction steps to enable ligation to other nucleic acids or nick-sealing. In some embodiments, fully double-stranded synthetic transposons having 5′ hydroxyl ends are used in strand displacement methods that fragment the target nucleic acids after insertion and gap-filling by a polymerase having strand displacement activity.

The synthetic transposons can be DNA, RNA, or a mixture of DNA and RNA. In some embodiments, the synthetic transposon comprises one or more modified nucleotides, such as locked nucleic acid (LNA) bases. Inclusion of modified nucleotides in the synthetic transposons may fine tune (such as increase or decrease) the binding stability between the transposase and the synthetic transposon, and/or minimize non-specific binding between the transposase and regions of the synthetic transposons outside the transposase recognition sites. In some embodiments, the synthetic transposon comprises 5-methyl dC, which is stable during bisulfite treatment. Synthetic transposons having 5-methyl dC may be particularly useful for barcoding target nucleic acids that are subject to sequencing analysis involving bisulfite treatment procedure, including, but not limited to, DNA (such as genome) methylation analysis, and sequencing or library construction methods that use bisulfite treatment to tag target nucleic acids via random mutagenesis (e.g., Levy D. and Wigler M., Proc. Natl. Acad. Sci. E4632-E4637, 2014).

The synthetic transposons provided herein can be prepared by a variety of methods. In some embodiments, the synthetic transposons are prepared by direct synthesis, including chemical synthesis. Such methods are well known in the art, e.g., solid phase synthesis using phosphoramidite precursors such as those derived from protected 2′-deoxynucleosides, ribonucleosides, or nucleoside analogues. Synthetic transposons comprising modified nucleotides (such as 5-methyl dC) may also be chemically synthesized by including modified nucleotide building blocks in the oligo synthesis steps. Alternatively, for synthetic transposons having a 5-methyl C in a CpG sequence, an unmodified synthetic transposon may first be synthesized, and the 5-methyl group may be added to the target dC nucleobase using a CpG methyltransferase. In some embodiments, the synthetic transposons are prepared by annealing two oligos, which are then subjected to extension by polymerases to provide the full product. Synthetic transposons with one or two hairpin structures can be conveniently prepared using a single long strand of oligonucleotide with complementary regions that hybridize to provide the synthetic transposons. In some embodiments, the synthetic transposons are PCR amplified with common primers, such as primers that hybridize to the additional sequences flanking the transposase recognition sites to prepare the synthetic transposons.

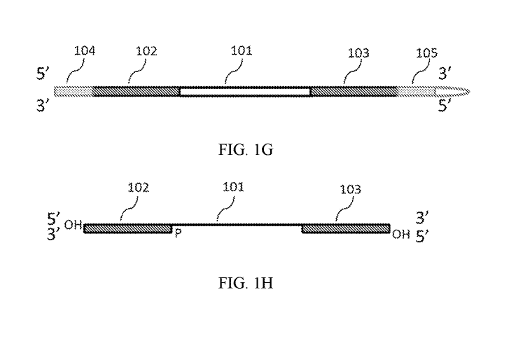

An example of a fully double-stranded synthetic transposon having Tn5 transposase recognition sites is shown in FIG. 2A. The transposase recognition sites are 19-bp inverted repeat sequences (201 and 202 of FIG. 2A), which flank the molecular barcode (203 of FIG. 2A). The synthetic transposon can be prepared from oligonucleotides (“oligos”), which can be chemically synthesized and obtained from many commercial manufacturers. For example, the exemplary synthetic transposon can be prepared from a long oligo with a “random” (i.e. randomly designed) molecular barcode (204) containing an intact 19-bp of Tn5 transposase recognition site on one end, and a short oligo with fixed bases, but truncated or no bases of the transposase recognition site (205) on the other end. With this exemplary preparation method, hairpin formation between the 19-bp inverted repeat sequences of the transposase recognition sequences can be minimized during the preparation process. Fixed nucleotides between randomly designed nucleotides (i.e., Ns) or degenerated nucleotides in the molecular barcodes can be carefully selected to minimize formation of hairpin structures within the molecular barcode sequences. After annealing the long and short oligos (204 and 205), buffer, dNTPs and a DNA polymerase are added to make the plurality of fully double-stranded synthetic transposons. Any leftover single-stranded oligos can be removed by treatment with Exonuclease I or other single-stranded specific nucleases. Any unwanted short double stranded products can be removed by standard nucleic acid purification methods (e.g., gel electrophoresis, column chromatography, or beads-based batch purification methods). In some embodiments, by choosing the right fixed nucleotides or nucleotides from lower degenerate sets (e.g., Y instead of N), the degenerately designed nucleotides and fixed nucleotides in the molecular barcodes minimize primer-dimer formation, and avoid accidental representation of another transposase recognition site sequence in the randomly designed barcode region.

An example of a synthetic transposon comprising a first transposase recognition site of Tn5 (a mosaic element, ME1, 201b), a second transposase recognition site of Tn5 (the inverse repeat of a mosaic element, ME2, 202b), and a partially single-stranded molecular barcode (203b) comprising 15 randomly designed nucleotides mixed with degenerately designed nucleotides and fixed nucleotides disposed therebetween in shown in FIG. 2B. Use of extra fixed bases between the transposase recognition sites allows easy generation of the double-stranded synthetic DNA with single-stranded molecular barcode sequence from 3 oligonucleotides (204b and 205b and 206b). Oligonucleotide 206b has a 5′-terminal nucleotide with a 5′ phosphate group. After hybridizing the oligonucleotide 206b with the long oligonucleotide 204b (shown here in linear format), initial extension displaces the internal hairpin structure of 204b. After removal of DNA polymerase or dNTPs, another oligonucleotide 205b is hybridized to the complex of 206b and 204b, resulting in the final synthetic transposon (top). Unused single stranded DNA can be removed by Exonuclease I or purified away from the desired dsDNA synthetic transposon.

### Methods of Library Preparation

One aspect of the present application provides a method of preparing a library of template nucleic acids comprising contacting (such as in vitro or in vivo) a target nucleic acid with any one of the compositions described herein and a transposase or integrase under a condition that allows insertion of at least a portion of the plurality of synthetic transposons into the target nucleic acid to provide a barcoded target nucleic acid. Further provided are barcoded target nucleic acids comprising a plurality of any of the synthetic transposons described herein.

Thus, for example, in some embodiments, there is provided a method (also referred herein as “non-strand displacement method”) of preparing a library of template nucleic acids, comprising: (a) contacting a target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; and (d) fragmenting the plurality of amplified barcoded target nucleic acids thereby providing the library of template nucleic acids. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the target nucleic acid is contacted with the plurality of synthetic transposons and the transposase in vitro. In some embodiments, the plurality of synthetic transposons and the transposase are pre-mixed prior to contacting the target nucleic acid. In some embodiments, the target nucleic acid is contacted with the plurality of synthetic transposons and the transposase in vivo. In some embodiments, the target nucleic acid is selected from the group consisting of cDNA, genomic DNA, bisulfite-treated DNA, and crosslinked DNA. In some embodiments, the plurality of synthetic transposons is inserted into the target nucleic acid at a frequency of at least once per about 500 bases (such as at least once per about 250 bases, or at least once per about 150 bases). In some embodiments, the method further comprises diluting the barcoded target nucleic acid into a plurality of compartments (such as wells in a plate). In some embodiments, the amplifying is PCR amplification. In some embodiments, the amplifying is whole genome amplification (WGA), for example, using random primers. In some embodiments, the amplifying is exome amplification using exome capture probes. In some embodiments, the method further comprises adaptor ligation prior to the amplifying.

In some embodiments, there is provided a method (also referred herein as “strand displacement method”) of preparing a library of template nucleic acids, comprising: (a) contacting a target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; and (c) amplifying the fragments to provide the library of template nucleic acids. In some embodiments, the target nucleic acid is contacted with the plurality of synthetic transposons and the transposase in vitro. In some embodiments, the plurality of synthetic transposons and the transposase are pre-mixed prior to contacting the target nucleic acid. In some embodiments, the target nucleic acid is contacted with the plurality of synthetic transposons and the transposase in vivo. In some embodiments, the target nucleic acid is selected from the group consisting of cDNA, genomic DNA, bisulfite-treated DNA, and crosslinked DNA. In some embodiments, the plurality of synthetic transposons is inserted into the target nucleic acid at a frequency of at least once per about 500 bases (such as at least once per about 250 bases, or at least once per about 150 bases). In some embodiments, the method further comprises diluting the barcoded target nucleic acid into a plurality of compartments (such as wells in a plate). In some embodiments, the amplifying is PCR amplification. In some embodiments, the amplifying is whole genome amplification (WGA), for example, using random primers. In some embodiments, the amplifying is exome amplification using exome capture probes. In some embodiments, the method further comprises adaptor ligation prior to the amplifying.

In some embodiments, there is provided a method (also referred herein as “combination method”) of preparing a library of template nucleic acids, comprising: (a) contacting a target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; and (d) amplifying the fragments to provide the library of template nucleic acids. In some embodiments, the target nucleic acid is contacted with the plurality of synthetic transposons and the transposase in vitro. In some embodiments, the plurality of synthetic transposons and the transposase are pre-mixed prior to contacting the target nucleic acid. In some embodiments, the target nucleic acid is contacted with the plurality of synthetic transposons and the transposase in vivo. In some embodiments, the target nucleic acid is selected from the group consisting of cDNA, genomic DNA, bisulfite-treated DNA, and crosslinked DNA. In some embodiments, the plurality of synthetic transposons is inserted into the target nucleic acid at a frequency of at least once per about 500 bases (such as at least once per about 250 bases, or at least once per about 150 bases). In some embodiments, the method further comprises diluting the barcoded target nucleic acid into a plurality of compartments (such as wells in a plate). In some embodiments, the amplifying is PCR amplification. In some embodiments, the amplifying is whole genome amplification (WGA), for example, using random primers. In some embodiments, the amplifying is exome amplification using exome capture probes. In some embodiments, the method further comprises adaptor ligation prior to the amplifying.

In some embodiments, there is provided a barcoded target nucleic acid comprising a plurality of synthetic transposons inserted randomly or substantially randomly among the endogenous sequence of the barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region. In some embodiments, there is provided a barcoded target nucleic acid comprising a plurality of synthetic transposons inserted randomly or substantially randomly among the endogenous sequence of the barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode. In some embodiments, each synthetic transposon is flanked by a pair of duplicated sequences endogenous to the barcoded target nucleic acid. In some embodiments, the first transposase recognition site is different from the second transposase recognition site. In some embodiments, the first transposase recognition site is the same as the second transposase recognition site. In some embodiments, the first transposase recognition site and/or the second transposase recognition site each comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA). In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated. In some embodiments, wherein each synthetic transposon comprises two double-stranded ends with no terminal hairpin structures, the 5′ termini of the two double-stranded ends are unphosphorylated, and the 5′ terminus adjacent to the single-stranded region is phosphorylated.

In some embodiments, there is provided a cell comprising a barcoded target nucleic acid comprising a plurality of synthetic transposons inserted randomly or substantially randomly among the endogenous sequence of the barcoded target nucleic acid, wherein each synthetic transposon comprises a first transposase recognition site, a second transposase recognition site, and a molecular barcode (such as partially single-stranded, single-stranded, or double-stranded) disposed between the first transposase recognition site and the second transposase recognition site, and wherein each synthetic transposon comprises a different molecular barcode. In some embodiments, each synthetic transposon is flanked by a pair of duplicated sequences endogenous to the barcoded target nucleic acid. In some embodiments, the plurality of synthetic transposons is inserted into the target nucleic acid at a frequency of at least once per about 500 bases (such as at least once per about 250 bases, or at least once per about 150 bases). In some embodiments, the first transposase recognition site is different from the second transposase recognition site. In some embodiments, the first transposase recognition site is the same as the second transposase recognition site. In some embodiments, the first transposase recognition site and/or the second transposase recognition site each comprise a mosaic element (ME). In some embodiments, the molecular barcode comprises at least about 5 (such as at least about any one of 10, 15, 20, or 25) randomly and/or degenerately designed nucleotides. In some embodiments, each synthetic transposon comprises one or more deoxyribonucleotides, ribonucleotides, or modified nucleotides (such as 5-methyl dC, or LNA). In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the 5′ termini of the two double-stranded ends are unphosphorylated. In some embodiments, wherein each synthetic transposon comprises two double-stranded ends with no terminal hairpin structures, the 5′ termini of the two double-stranded ends are unphosphorylated, and the 5′ terminus adjacent to the single-stranded region is phosphorylated.

The plurality of synthetic transposons can be inserted into target nucleic acids either in vitro or in vivo by the transposase that binds to the transposase recognition sites of the synthetic transposons. For in vitro insertion methods, the plurality of synthetic transposons and the transposase may be pre-mixed to form a complex composition comprising a plurality of complexes each comprising a transposase bound to a synthetic transposon prior to contacting the complex composition with the target nucleic acid. In some embodiments, the plurality of synthetic transposons and the transposase are contacted with the target nucleic acids simultaneously, but as separate compositions. For in vivo insertion methods, the plurality of synthetic transposons and a nucleic acid (such as a viral vector or a plasmid) encoding the transposase can be transfected or transduced into a cell having the target nucleic acid to allow contact of the transposase-synthetic transposon complex with the target nucleic acid. The barcoded target nucleic acid can be subsequently isolated from the cell and used as templates to construct a sequencing library.

In some embodiments, synthetic transposons with molecular barcodes having high diversity, for example, comprising more than about any one of 5, 10, 15, 20, 25, or more randomly and/or degenerately designed nucleotides are used to ensure that each insertion site in the target nucleic acid has a different molecular barcode. In some embodiments, an excess amount of synthetic transposons is contacted with the target nucleic acid to ensure unique labeling of the sites in the target nucleic acid. In some embodiments, no more than about any one of 50%, 40%, 30%, 20%, 10%, 5%, 2%, 1%, 0.1%, 0.01%, 0.001%, 0.0001% or less of possible synthetic transposons with distinct molecular barcodes are inserted into the target nucleic acid. For example, 100 cells of human genomic DNA (about 0.6 ng) have a total of 300×10⁹ basepairs. After insertion of synthetic transposons each having a molecular barcode comprising 25 randomly designed nucleotides at an average of 150-bp distance, a total of 2×10⁹ synthetic transposons are inserted out of 10¹⁵ possible distinct synthetic transposons available. Thus, there is a 1 in 500,000 chance to have identical synthetic transposons at two different sites in the barcoded genomic DNA. By combining the transposase duplicated sequences (e.g., 9-nt duplicate sequence of Tn5 transposase) and the molecular barcode sequences, it would be easy to differentiate and align sequencing reads derived from neighboring fragments in a single target molecule.

As used herein, the term “at least a portion” or grammatical equivalents thereof can refer to any fraction of a whole amount. For example, “at least a portion” can refer to at least about any one of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 99.9% or 100% of a whole amount. In some embodiments, at least about any one of 10%, 20%, 30%, 40%, 50%, 60%, 70%, 80%, 90%, 95%, 99% or more of the plurality of synthetic transposons is inserted in the target nucleic acid.

The frequency (i.e., density) of the synthetic transposons inserted in the target nucleic acid can be controlled by various ways, including adjusting the contacting time and temperature, the amount of synthetic transposons, the type and amount of the transposase, and composition of the buffer. In some embodiments, the plurality of synthetic transposons are inserted into the target nucleic acid at a frequency of at least once per about any one of 10 kb, 5 kb, 4 kb, 3 kb, 2 kb, 1 kb, 900 bases, 800 bases, 700 bases, 600 bases, 500 bases, 400 bases, 300 bases, 250 bases, 200 bases, 150 bases, 100 bases, or fewer. In some embodiments, the plurality of synthetic transposons are inserted into the target nucleic acid at a frequency of once per any one of about 100 bases to about 200 bases, about 150 bases to about 250 bases, about 250 bases to about 500 bases, about 500 bases to about 750 bases, about 750 bases to about 1 kb, about 1 kb to about 5 kb, about 5 kb to about 10 kb, about 100 bases to about 1 kb, or about 100 bases to about 10 kb.

It should be recognized by persons skilled in the art that there is an increased sequencing cost associated with an increased density of synthetic transposon insertion. Insertion with 75-nucleotide synthetic transposons at a once per about 150 bases frequency results in about 50% higher cost based on the number of bases need to be sequenced. By contrast, a barcoded target nucleic acid with the same synthetic transposons and an insertion frequency of once per about 300 bases results in about 25% higher sequencing cost than sequencing the non-barcoded target nucleic acid. Therefore, a tradeoff between sequencing cost and quality may be considered when using libraries prepared with the methods described herein. For example, synthetic transposons described herein may be particularly useful and effective for preparing sequencing libraries for whole genome sequencing requiring high quality (for example, error rate lower than about 1 in 10⁶ bases), targeted capture sequencing, or microbiome sequencing in clinical setting. With advancements in sequencing technologies, the sequencing cost per base has been dropping and we expect that per base sequencing cost will not be the main cost for many of the applications described herein in the future.

The target nucleic acid can include any nucleic acid of interest. Target nucleic acids can include, DNA, RNA, peptide nucleic acid, morpholino nucleic acid, locked nucleic acid, glycol nucleic acid, threose nucleic acid, mixtures thereof, and hybrids thereof. In some embodiments, the target nucleic acid is genomic DNA, such as whole genome, part of the genome (e.g., individual chromosomes or fragments thereof), mixed genomes (e.g., microbiome). Intact chromosomes in live cells or isolated intact chromosomes can be used to achieve longest contiguity contigs as possible for any given species. Careful isolation of intact chromosomes has been demonstrated previously (e.g., Howe B. et al., Chromosome preparation from cultured cells. J Vis. Exp. 83: e50203, 2014). In some embodiments, the target nucleic acid is mitochondrial DNA. In some embodiments, the target nucleic acid is chloroplast DNA. In some embodiments, the target nucleic acid is cDNA, synthetic or modified DNA after certain chemical or enzymatic treatments, including bisulfite treatment (e.g., for CpG methylation detection).

The target nucleic acid can be of any length. The synthetic transposons and the methods described herein are particularly useful for preparing barcoded libraries to be sequenced and assembled to analyze long, contiguous target nucleic acids having a length of at least about any one of 10 kb, 20 kb, 50 kb, 100 kb, 200 kb, 500 kb, 1 Mb, 2 Mb, 5 Mb, 10 Mb, 20 Mb, 50 Mb, 100 Mb, 200 Mb, or more. The target nucleic acid can comprise any nucleotide sequences. In some embodiments, the target nucleic acid comprises homopolymer sequences. The target nucleic acid can also include repeat sequences. Repeat sequences can be any of a variety of lengths including, for example, at least about any one of 2, 5, 10, 20, 30, 40, 50, 100, 250, 500, 1000 nucleotides or more. Repeat sequences can be repeated, either contiguously or non-contiguously, any of a variety of times including, for example, at least about any one of 2, 3, 4, 5, 6, 7, 8, 9, 10, 15, 20 times or more.

In some embodiments, the plurality of synthetic transposons is inserted in a single target nucleic acid. In some embodiments, the plurality of synthetic transposons is inserted in a plurality of target nucleic acids. In such embodiments, a plurality of target nucleic acids can include a plurality of the same target nucleic acids, a plurality of different target nucleic acids wherein some target nucleic acids are the same, or a plurality of target nucleic acids wherein all target nucleic acids are different. Embodiments that involve a plurality of target nucleic acids can be carried out in multiplex formats such that reagents can be delivered simultaneously to the target nucleic acids, for example, in one or more compartments or on an array surface. In some embodiments, the plurality of target nucleic acids can include substantially all of a particular organism's genome. The plurality of target nucleic acids can include at least a portion of a particular organism's genome, including, for example, at least about any one of 1%, 5%, 10%, 25%, 50%, 75%, 80%, 85%, 90%, 95%, or 99% of the genome. In particular embodiments, the portion can have an upper limit that is at most about any one of 1%, 5%, 10%, 25%, 50%, 75%, 80%, 85%, 90%, 95%, or 99% of the genome.

Target nucleic acids can be obtained from any source. For example, target nucleic acids may be prepared from nucleic acid molecules obtained from a single organism or from populations of nucleic acid molecules obtained from natural sources that include one or more organisms. Sources of nucleic acid molecules include, but are not limited to, organelles, cells, tissues, organs, or organisms. Cells that may be used as sources of target nucleic acid molecules may be prokaryotic (bacterial cells, for example, Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces genera); archeaon, such as crenarchaeota, nanoarchaeota or euryarchaeotia; or eukaryotic such as fungi, (for example, yeasts), plants, protozoans and other parasites, and animals (including insects (for example, Drosophila spp.), nematodes (for example, Caenorhabditis elegans), and mammals (for example, rat, mouse, monkey, non-human primate and human)).

In some embodiments, a transposase (such as Tn5 transposase) binds the transposase recognition sites, makes staggered cuts at random sites in a target nucleic acid, and inserts synthetic transposons at the cut sites, resulting in a pair of single-stranded gaps of a fixed length flanking the inserted synthetic transposon sequence in the target nucleic acid. The single-stranded gaps have duplicated sequences derived from the target nucleic acid. The duplicated sequences are characteristic for each transposase, for example, the duplicated sequences are 9-nt long for Tn5 transposase, 5-nt long for Tn7 and Mu transposases, 4-nt long for murine leukemia virus, and 2-nt long for Tc1/marine family. Transposition events are random or substantially random. For example, some studies show certain transposition biases (see, e.g., Green B et al, “Insertion site preference of Mu, Tn5, and Tn7 transposons” Mobile DNA 3:3, 2012).

Once synthetic transposons are integrated into target nucleic acids, there are several ways to keep the contiguity (e.g., haplotyping) information as tagged by the distinct molecular barcodes. The target nucleic acids inserted with the synthetic transposons can be repaired with a polymerase without strand displacement activity and a ligase in vitro or in vivo so the synthetic transposons can be an integrated part of the target nucleic acids. The polymerase without strand displacement activity allows gap filling of any single-stranded nucleic acid created surrounding the insertion sites (such as single-stranded gaps having duplicated sequences endogenous to the target nucleic acid). The ligase allows nick sealing for nicks having a 5′ phosphate. The gap filling reaction catalyzed by the polymerase without strand displacement, and the ligation reaction catalyzed by the ligase can be carried out in a single step, or in separate steps comprising first contacting the target nucleic acid inserted with the synthetic transposons with the polymerase without strand displacement activity and nucleotides, followed by contacting the resulting product with the ligase.

Alternatively, after transposition, a polymerase with strand displacement activity can be used to fill in the single-stranded gaps and displace one of the synthetic transposon's strands to generate identical transposon sequences on one end of each of the two fragments generated thereof. All fragments generated thereof, except for the fragments at each end of the target nucleic acid, have a first synthetic transposon on one end and a second synthetic transposon on the other end. Fragments derived from neighboring positions in the target nucleic acid share the same synthetic transposon at the contiguous ends. These fragments can be further amplified, captured with specific probes if needed, and sequenced using current next generation sequencing technologies.

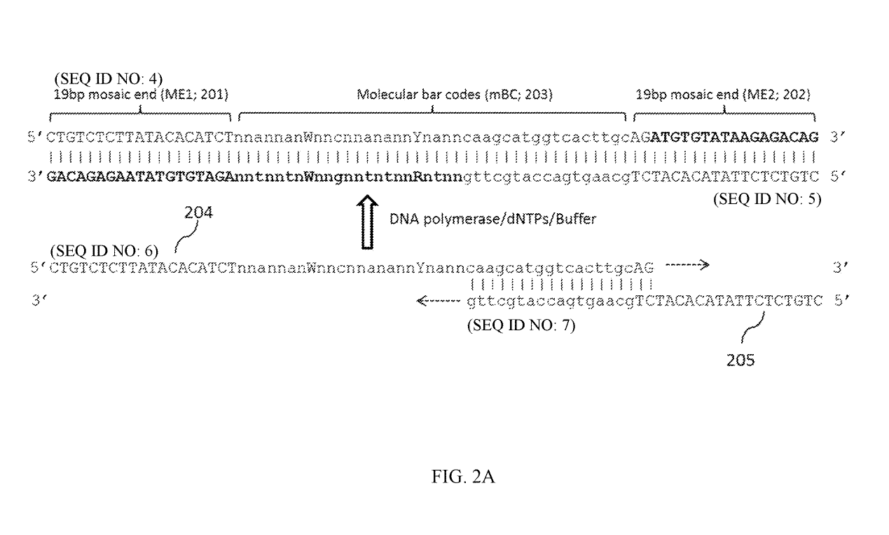

FIG. 3 shows a schematic example of transposition of a synthetic transposon (ME1+mBC+ME2, as shown in FIG. 2) into a double stranded genomic DNA by Tn5 transposase. Tn5 binds the mosaic ends of the synthetic transposon and forms a dimeric complex. Random transposition of each Tn5/synthetic transposon complex into the target gDNA results in staggered cut of a 9-bp sequence of the target gDNA at the insertion site, yielding a 9-nt single-stranded gap on each side of the inserted synthetic transposon. When molecular barcodes with high diversity (for example, achieved by the use of ˜25 randomly designed nucleotides or 10¹⁵ possible sequences) are used, each mBC integrated within the target nucleic acid is different.

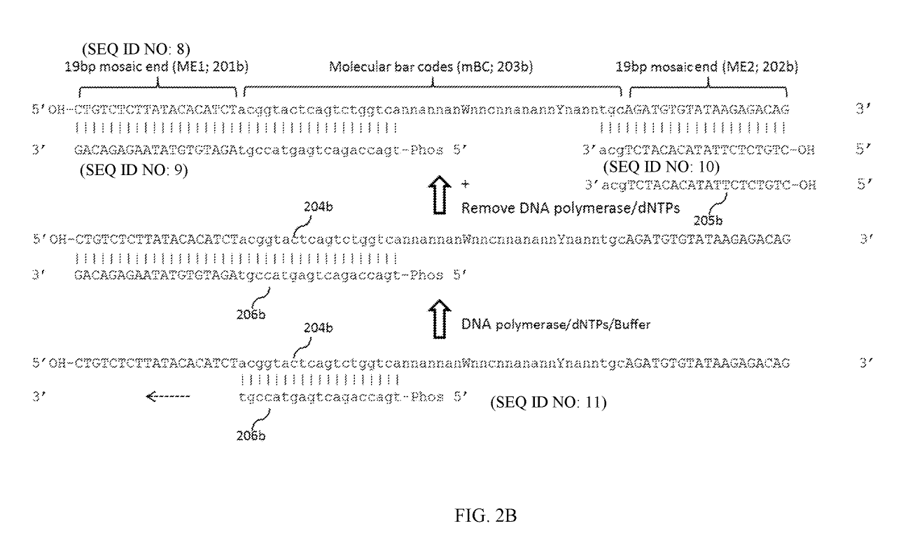

FIG. 4 shows an exemplary strand displacement method for preparing a library of template nucleic acids after insertion of synthetic transposons by Tn5 transposase (e.g., as shown in FIG. 3). In step (a) of FIG. 4, 9-nt single-stranded gaps are created during transposition catalyzed by Tn5, which can be filled in by a DNA polymerase in the presence of dNTPs and buffer as shown in step (b). Using a DNA polymerase with strand displacement activity, the enzyme can extend the synthesis and displace one strand of the synthetic transposon (step (c) of FIG. 4) until separation of the original synthetic transposon strands and completion of the gap fill-in (step (d) of FIG. 4). Consequently, the sequence of the synthetic transposon is duplicated. The resulting adjacent DNA fragments each have the sequence of the synthetic transposon and the 9-nt gap on one end, and the molecular barcode sequence in the synthetic transposons on such ends are identical to each other. The resulting template DNA fragments can be amplified after end repair and ligation to adaptors to prepare a sequencing library. The molecular barcodes can thus be used to cluster and link sequencing reads sharing the same molecular barcodes to derive the contiguous sequences of the original target molecules with haplotype information preserved. No additional restriction digestion, or any other fragmentation or modification steps are required in such workflow. The duplicated 9 nt gap sequences next to the synthetic transposons can be further used to facilitate the clustering algorithm to “stitch” or link the fragments together and to derive the contiguous sequence of a long, contiguous target nucleic acid. It is noted that synthetic transposons having either single-stranded molecular barcodes or double-stranded molecular barcodes may be used in this exemplary workflow.

For synthetic transposons having partially or fully single-stranded molecular barcodes, synthetic transposons as shown in FIG. 1H or FIG. 2B may be used in a combination method for library preparation. In such case, after insertion of the synthetic transposons, a polymerase without strand displacement activity (such as T4 DNA polymerase) and nucleotides (such as dNTPs) can be used to fill in the single-stranded gaps, and a ligase can be used to seal the nick inside the synthetic transposon sequence. Then A DNA polymerase with strand displacement can be used to generate fragment with ends having identical sequences of the synthetic transposons, such as in step (c) of FIG. 4.

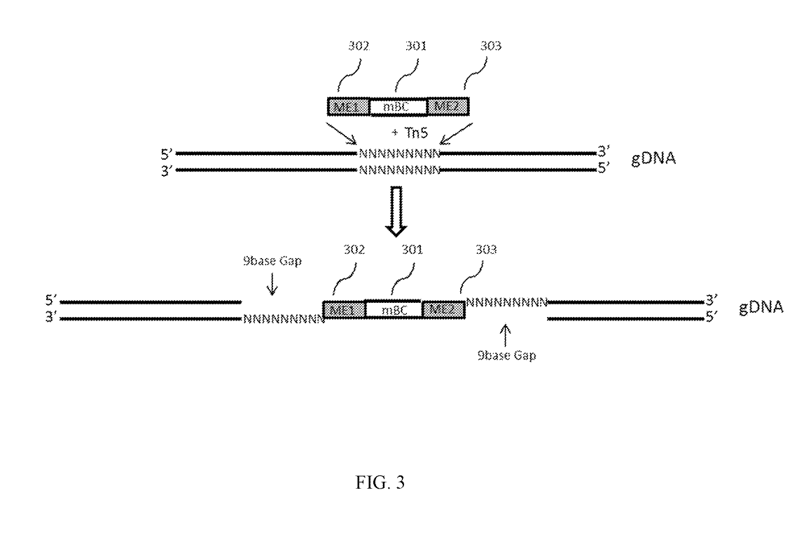

FIG. 5 shows an exemplary non-strand displacement method for preparing a library of template nucleic acids while keeping contiguity information after insertion of synthetic transposons into target nucleic acids, which is repaired without breaking the target nucleic acids. As shown in steps (a)-(b) of FIG. 5, the DNA template is inserted with synthetic transposons at multiple random sites, followed by gap fill-in with dNTPs and DNA polymerase without strand displacement activity, while the resulting nicks are sealed by a ligase. The resulting DNA is amplified, for example, through multiple displacement amplification (i.e., “MDA”) using kits such as GenomiPhi™ (GE Health) or Repli-g™ (Qiagen) in step (c). This amplification step allows preparation of multiple copies (usually thousands to millions) of template DNAs with the same molecular barcodes. Errors and bias from this amplification step can be easily corrected by deriving consensus sequences from the template DNAs having the same molecular barcodes. The amplified DNA is then fragmented by mechanical (e.g., ultrasonic) or enzymatic (e.g., DNase I) methods in step (d) and used for sequencing after library construction in step (e).

In some embodiments, the method comprises amplification (such as PCR amplification) of the barcoded target nucleic acids or fragments thereof. For example, primers that hybridize to the transposase recognition sites and optionally additional fixed sequences surrounding the randomly or degenerately designed molecular barcode sequences (e.g., for better specificity and adaptor-index sequences) can be used for the amplification. In some embodiments, tandem primers may also be used for whole genome amplification. In some embodiments, primers that selectively hybridize to sequences of interest, such as exome probes, may be used for amplification of targeted sequences. In some embodiments, adaptors and/or sample tags may be ligated to the fragments prior to the amplification. The amplification step may need long annealing/extension time to obtain products of appropriate size. The method may further comprise purification step(s) to remove short, unwanted products with only the transposon sequences.

In some embodiments, the method may comprise a dilution step to separate the nucleic acid sample, such as the target nucleic acid, the barcoded target nucleic acid, or the repaired barcoded target nucleic acid into a plurality of compartments (such as wells in a multi-well plate). In some embodiments, the nucleic acid sample is diluted into at least about any of 5, 10, 20, 50, 100, 200, 300, 500 or more compartments to allow subsequent steps, such as amplification, in the methods to carry out within the individual compartments. In some embodiments, each compartment comprises no more than about any of 5000, 1000, 500, 200, 100, 50, 20, 10, 5, or fewer molecules. Compartment tags may be introduced to the template nucleic acid in the adaptor ligation or amplification step. Samples from the compartment can be pooled together during sequencing, and the sequencing reads may be de-multiplexed using the compartment tags. The dilution may facilitate mapping of sequencing reads to individual target nucleic acids or segments thereof.

### Methods of Analysis

The present application further provides methods of analyzing a target nucleic acid by sequencing libraries of template nucleic acids prepared using any of the methods described above.

In some embodiments, there is provided a method of analyzing a target nucleic acid, or sequencing (such as next-generation sequencing or massively parallel sequencing) a target nucleic acid, comprising: (a) preparing a library of template nucleic acids from the target nucleic acid using any one of the methods described in the “Methods of library preparation” section; (b) sequencing the library of template nucleic acids to obtain sequencing reads; and (c) assembling a contiguous sequence of the target nucleic acid from the sequencing reads based on the molecular barcodes of the synthetic transposons in the template nucleic acids. In some embodiments, the sequencing is massively parallel shotgun sequencing. In some embodiments, step (c) comprises (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing a target nucleic acid, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; and (d) fragmenting the plurality of amplified barcoded target nucleic acids thereby providing a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing a target nucleic acid, comprising: (a) contacting a target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing a target nucleic acid, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; and (e) assembling a contiguous sequence of the target nucleic acid from the sequencing reads based on the molecular barcodes of the synthetic transposons in the template nucleic acids. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of sequencing (such as next-generation sequencing or massively parallel sequencing) a target nucleic acid, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of sequencing (such as next-generation sequencing or massively parallel sequencing) a target nucleic acid, comprising: (a) contacting a target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of sequencing (such as next-generation sequencing or massively parallel sequencing) a target nucleic acid, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; and (e) assembling a contiguous sequence of the target nucleic acid from the sequencing reads based on the molecular barcodes of the synthetic transposons in the template nucleic acids. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of insertion of random synthetic transposon (RST) randomly in targeted nucleic acids in vivo or in vitro to allow whole genome, chromosome or long range haplotyping, sequencing and accurate quantitation of desired nucleic acids, said method comprising: (a) Mix or pre-mix the transposase with the random synthetic transposon (RST) to form transposon complex; (b) Mix the transposon complex with target nucleic acids to allow the random or near random insertion of the RST; (c) Repair the insertion site with DNA polymerase with dNTPs and a buffer and with or without ligase; (d) Dilute or aliquot to multiple wells if needed; (e) Amplify the target nucleic acids integrated with RSTs with methods such as PCR after adaptor ligation or displacement amplification followed by fragmentation/adaptor ligation/PCR amplification; (f) Perform sequencing such as next generation sequencing to obtain raw data or selected sequences can be captured for exome or targeted sequencing; and (g) Input to a software program for analysis. In some embodiments, the target nucleic acid is originally from cDNA, genomic DNA or modified DNA such as bisulfite-treated genomic DNA for methylation status. In some embodiments, the nucleic acids could be treated with crosslinking chemicals such as formaldehyde to maintain the chromosome in a native 3-D structure to assess the compartmentalization of the genome. In some embodiments, the gapped region at the insertion site is filling-in with dNTPs and DNA polymerase without displacement activity and nick ligated to repair the targeted DNA intact followed by random fragmentation (other than the Nextera system using Tn5) to construct a library for massive parallel shotgun sequencing. In some embodiments, the transposed target nucleic acids with double stranded RST are filling in by dNTPs and DNA polymerase with strand displacement activity resulting duplication of the original transposons with distinct barcodes, then end repaired and attached to common adaptor sequence and sample tags for amplification and sequencing.

### Sequencing

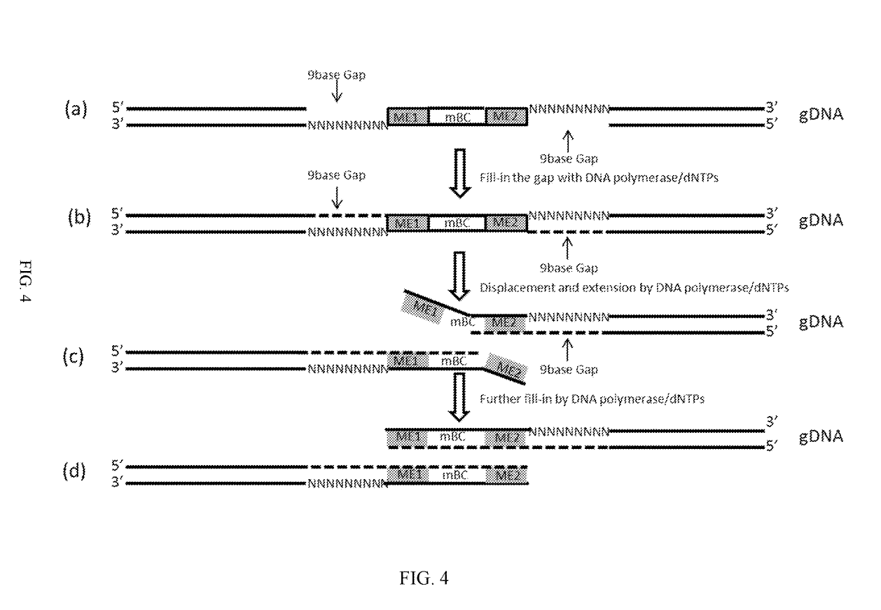

The methods described herein may comprise any one or more of library construction steps known in the art to prepare a sequencing library from the library of template nucleic acids, including, but not limited to, end repair, ligation to adaptors, amplification, sample tag addition, etc. FIG. 6 shows an exemplary method of library construction from short double-stranded DNA fragments such as the ones produced in step (d) of FIG. 4 or step (d) of FIG. 5. The fragments (601) can be first repaired to provide fragments with blunt ends (602), and subject to addition of dA (603), followed by ligation to adaptors (604) to provide a ligated product (605) that allows amplification with platform-dependent common primers and optional sample tags to obtain the final library constructs (606) ready for sequencing. In some embodiments, the library construction method comprises an exome capture step.

The methods described herein can be used in conjunction with a variety of sequencing techniques and platforms. In some embodiments, the process to determine the nucleotide sequence of a target nucleic acid can be an automated process. In some embodiments, the sequencing method is a massively parallel shotgun sequencing method. In some embodiments, the sequencing method yields short sequencing reads, such as sequencing reads of no more than about any one of 500 bases, 400 bases, 300 bases, 250 base, 200 bases, 150 bases, 100 bases, or fewer. Exemplary sequencing platforms include, but are not limited to, Roche 454 platforms, Illumina HiSeq, MiSeq, and NextSeq platforms, Life Technologies SOLiD platforms, Ion Torrent platforms, and Pacific Biosciences and PacBio RS platforms.

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568 and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons.

In another example type of sequence by sequencing (SBS) techniques, cycle sequencing is accomplished by stepwise addition of reversible terminator nucleotides containing, for example, a cleavable or photobleachable dye label as described, for example, in U.S. Pat. No. 7,427,67, U.S. Pat. No. 7,414,1163 and U.S. Pat. No. 7,057,026, the disclosures of which are incorporated herein by reference. This approach is being commercialized by Solexa (now Illumina Inc.), and is also described in WO 91/06678 and WO 07/123,744 (filed in the United States patent and trademark Office as U.S. Ser. No. 12/295,337), each of which is incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Additional example SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199 and PCT Publication No. WO 07/010,251, the disclosures of which are incorporated herein by reference in their entireties.

Some embodiments can utilize sequencing by ligation techniques. Such techniques utilize DNA ligase to incorporate short oligonucleotides and identify the incorporation of such short oligonucleotides. Example SBS systems and methods which can be utilized with the methods and systems described herein are described in U.S. Pat. No. 6,969,488, U.S. Pat. No. 6,172,218, and U.S. Pat. No. 6,306,597, the disclosures of which are incorporated herein by reference in their entireties.

Some embodiments can include techniques such as next-next technologies. One example can include nanopore sequencing techniques (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). In some such embodiments, nanopore sequencing techniques can be useful to confirm sequence information generated by the methods described herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. No. 7,329,492 and U.S. Pat. No. 7,211,414 (each of which is incorporated herein by reference in their entireties) or nucleotide incorporations can be detected with zero-mode waveguides as described, for example, in U.S. Pat. No. 7,315,019 (which is incorporated herein by reference in its entirety) and using fluorescent nucleotide analogs and engineered polymerases as described, for example, in U.S. Pat. No. 7,405,281 and U.S. Patent Application Publication No. 2008/0108082 (each of which is incorporated herein by reference in their entireties). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nanostructures.” Proc. Natl. Acad. Sci. USA 105, 1176-1181 (2008), the disclosures of which are incorporated herein by reference in their entireties). In one example single molecule, real-time (SMRT) DNA sequencing technology provided by Pacific Biosciences Inc. can be utilized with the methods described herein. In some embodiments, a SMRT chip or the like may be utilized (U.S. Pat. Nos. 7,181,122, 7,302,146, 7,313,308, incorporated by reference in their entireties). A SMRT chip comprises a plurality of zero-mode waveguides (ZMW). Each ZMW comprises a cylindrical hole tens of nanometers in diameter perforating a thin metal film supported by a transparent substrate. When the ZMW is illuminated through the transparent substrate, attenuated light may penetrate the lower 20-30 nm of each ZMW creating a detection volume of about 1×10-21 L. Smaller detection volumes increase the sensitivity of detecting fluorescent signals by reducing the amount of background that can be observed.

SMRT chips and similar technology can be used in association with nucleotide monomers fluorescently labeled on the terminal phosphate of the nucleotide (Korlach J. et al., “Long, processive enzymatic DNA synthesis using 100% dye-labeled terminal phosphate-linked nucleotides.” Nucleosides, Nucleotides and Nucleic Acids, 27:1072-1083, 2008; incorporated by reference in its entirety). The label is cleaved from the nucleotide monomer on incorporation of the nucleotide into the polynucleotide. Accordingly, the label is not incorporated into the polynucleotide, increasing the signal: background ratio. Moreover, the need for conditions to cleave a label from a labeled nucleotide monomer is reduced.

An additional example of a sequencing platform that may be used in association with some of the embodiments described herein is provided by Helicos Biosciences Corp. In some embodiments, true single molecule sequencing can be utilized (Harris T. D. et al., “Single Molecule DNA Sequencing of a viral Genome” Science 320:106-109 (2008), incorporated by reference in its entirety). In one embodiment, a library of target nucleic acids can be prepared by the addition of a 3′ poly(A) tail to each target nucleic acid. The poly(A) tail hybridizes to poly(T) oligonucleotides anchored on a glass cover slip. The poly(T) oligonucleotide can be used as a primer for the extension of a polynucleotide complementary to the target nucleic acid. In one embodiment, fluorescently-labeled nucleotide monomer, namely, A, C, G, or T, are delivered one at a time to the target nucleic acid in the presence DNA polymerase. Incorporation of a labeled nucleotide into the polynucleotide complementary to the target nucleic acid is detected, and the position of the fluorescent signal on the glass cover slip indicates the molecule that has been extended. The fluorescent label is removed before the next nucleotide is added to continue the sequencing cycle. Tracking nucleotide incorporation in each polynucleotide strand can provide sequence information for each individual target nucleic acid.

### Analysis

Sequencing reads can be analyzed with various methods. In some embodiments, an automated process, such as computer software, is used to analyze the sequencing reads to provide a contiguous sequence of the target nucleic acid. Analysis software can be developed from scratch or from current computational software to include mBC identification and clustering algorithms described herein for sequence assembly (de novo or using a reference).

In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; (iii) clustering the aligned sequencing reads based on the molecular barcodes in the synthetic transposons to provide the contiguous sequence. In some embodiments, wherein each synthetic transposon inserted in the target nucleic acid is flanked by a pair of single-stranded gaps having duplicated sequences endogenous to the target nucleic acid, step (ii) comprises aligning sequencing reads having the same molecular barcodes in the synthetic transposons and the same duplicated sequences of the single-stranded gaps to provide aligned sequencing reads, and/or step (iii) comprises clustering the sequencing reads based on the molecular barcodes in the synthetic transposons and the duplicated sequences of the single-stranded gaps. In some embodiments, step (iii) comprises deriving a contig from the clustered sequencing reads and removing the sequences of the synthetic transposons (and if applicable, one copy of the duplicated sequences of the single-stranded gaps) from the contig to provide the contiguous sequence. In some embodiments, the method further comprises counting one copy of the target nucleic acid for all sequencing reads assembled to the contiguous sequence.

In some embodiments, wherein the template nucleic acids each (except for those derived from the ends of the target nucleic acid) comprise a first synthetic transposon comprising a first molecular barcode at one end and a second synthetic transposon comprising a second molecular barcode at the other end (i.e., libraries prepared using any one of the strand-displacement methods or the combination methods described herein), the sequencing reads are assembled to provide a contiguous sequence of the target nucleic acid by steps comprising: (i) identifying sequences of the synthetic transposons in the sequencing reads; (ii) aligning sequencing reads having the same first molecular barcode and the same second molecular barcode; (iii) determining a consensus sequence for each group of aligned sequencing reads; (iv) linking the consensus sequences together based on the molecular barcodes in the synthetic transposons to provide a contig; and (v) removing the sequences of the synthetic transposons (and if applicable, one copy of the duplicated sequences of the single-stranded gaps) from the contig. In some embodiments, wherein each synthetic transposon inserted in the target nucleic acid is flanked by a pair of single-stranded gaps having duplicated sequences endogenous to the target nucleic acid, step (ii) comprises aligning sequencing reads having the same first molecular barcodes, the same second molecular barcodes, and the same duplicated sequences of the single-stranded gaps; and/or step (iv) comprises linking the consensus sequences together based on the molecular barcodes in the synthetic transposons and the duplicated sequences of the single-stranded gaps to provide the contig. In some embodiments, a consensus sequence is determined for each group having at least three aligned sequencing reads. In some embodiments, a mismatch nucleotide in a group of aligned sequencing reads is considered to be an amplification or sequencing error if no more than ⅓ or aligned sequencing reads in the group has the mismatch nucleotide. In some embodiments, the method further comprises counting one copy of the target nucleic acid for all sequencing reads assembled to the contiguous sequence.

In some embodiments, wherein the library of template nucleic acids prepared using any one of the non-strand displacement methods described herein, the sequencing reads are assembled to provide a contiguous sequence of the target nucleic acid by steps comprising: (i) identifying sequences of the synthetic transposons in the sequencing reads; (ii) aligning sequencing reads having the same molecular barcodes; (iii) clustering the aligned sequencing reads based on the molecular barcodes in the synthetic transposons; (iv) determining a contig of the clustered sequencing reads; and (v) removing the sequences of the synthetic transposons (and if applicable, one copy of the duplicated sequences of the single-stranded gaps) from the contig thereby providing the contiguous sequence of the target nucleic acid. In some embodiments, wherein each synthetic transposon inserted in the target nucleic acid is flanked by a pair of single-stranded gaps having duplicated sequences endogenous to the target nucleic acid, step (ii) comprises aligning sequencing reads having the same molecular barcodes and the same duplicated sequences of the single-stranded gaps; and/or step (iii) comprises clustering the aligned sequencing reads based on the molecular barcodes in the synthetic transposons and the duplicated sequences of the single-stranded gaps to provide the contig. In some embodiments, a mismatch nucleotide in the aligned sequencing reads is considered to be an amplification or sequencing error if no more than ⅓ of aligned sequencing reads covering the mismatched nucleotide position has the mismatch nucleotide. In some embodiments, the method further comprises counting one copy of the target nucleic acid for all sequencing reads assembled to the contiguous sequence.

In some embodiments, there is provided a software analysis algorithm and process to assembly the whole genome sequence or complete haplotyping information using RSTs and the duplicate sequences at insertion sites and obtain accurate counting of original molecules or copies of the sequences, comprising: a) demultiplex raw data to assign reads to each samples; b) align the reads for each sample; c) identify the first and second transposase recognition sequences separated by defined length in a particular RST used; d) cluster reads by the molecular bar code between the 2 transposase recognition sequences (exogenous molecular bar code) and the sequence next to the transposase recognition site (endogenous molecular bar code, e.g., 9-bp with Tn5) and correct any errors using combined bar codes; and e) generate final sequence for the genome or reports variants or copy number changes as needed. In some embodiments, reads with identical molecular bar code sequence and identical surrounding sequences including the 9-bp sequences previously seen can be removed as contamination. In some embodiments, reads with the same molecular bar code sequence merged as single molecule and base difference seen in a small portion of the reads can be corrected as amplification or sequencing errors. In some embodiments, variants such as indel or copy number changes or mutations (if a cancer library compared with a normal library) are identified and indexed.

In some embodiments, the sequencing data with the base calls and sample tag information are analyzed through a special pipeline to allow de-multiplexing of samples followed by clustering, error correction and assembly. Sequences of the transposase recognition sites can be used to identify the location of the synthetic transposons in the sequencing reads. In the cases of Tn5 synthetic transposons, a total of 38-bp Tn5 recognition sequences (2×19-bp, 4³⁸ or ˜7×10²² possibilities among 38-bp) separated by a fixed length of molecular barcode sequences, can be used quite uniquely for transposon identification in a large genome such as human (about 3×10⁹ bases). The fixed bases in the molecular barcode sequences can also serve as additional known bases for identification of the synthetic transposons among the sequencing reads. Once the transposons are identified, the distinct molecular barcode sequence between the transposase recognition sequences in a synthetic transposon (for example, a molecular barcode with 20 randomly designed nucleotides yields about 10¹² distinct sequences) can serve as exogenous tags. Additionally, when applicable, the duplicate gap sequences can serve as endogenous tags. For example, Tn5 generates 9-bp duplicated sequences (4⁹ or ˜2×10⁵ combinations) flanking the insertion sites, which provides information on the distinct positions of insertion. The duplicated gap sequence can provide additional insertion-specific information for mapping sequencing reads comprising the synthetic transposons to the original location in the target nucleic acid molecule. In embodiments with Tn5 synthetic transposons having 20 randomly designed nucleotides in the molecular barcodes, a total of greater than 2×10¹⁷ combinations of different sequences can theoretically be used for tagging and extracting contiguity information in a target nucleic acid. This large diversity of molecular barcodes allows the inserted sequences to be different in all positions. Therefore, each combination of exogenous and optionally endogenous tag sequences uniquely identifies the surrounding sequences from the target nucleic acid. The distinct molecular barcodes and the duplicate gap sequences from target nucleic acids on one or both ends of the synthetic transposon can serve as unique identifiers to cluster sequencing reads with the same molecular barcode and duplicated gap sequence. Amplification or sequencing errors are corrected and amplification bias is eliminated in the clustering process. Such methods can be particularly useful for assembling repetitive sequence regions, such as Alu repeats, so that the contiguity of the repetitive sequences can be resolved. Consensus sequences derived from the clustered reads are then assembled together to obtain a phased uninterrupted sequence for the target nucleic acid.

In analysis, several parameters can be used to help cluster and assemble the sequencing reads to obtain maximal haplotyping information and lead to final counting of the original target molecules. For example, the synthetic transposons can be identified using the 2 transposase recognition sequences (2×19-bp for Tn5 transposase recognition sites). Then the randomly designed sequences in the molecular barcodes (exogenous tags) and/or the duplicate gap sequences flanking the synthetic transposon insertion position (endogenous tags; e.g., 9-nt for Tn5 transposase, which yields 4⁹ possible sequences) can be used to trace back the original position of the insertion site in the target nucleic acid and count the original target nucleic acid once for each cluster of reads mapping to the same original target nucleic acid. Although it is possible to only use the molecular barcode sequences in the synthetic transposons, use of the duplicated gap sequences can provide additional information for assembly of the sequencing reads. For target nucleic acids in homogenous samples, the overlapped sequences among different clustered reads should be the same except for errors from amplification, and/or sequencing, and/or analysis steps. Therefore, a contig representing the error-corrected consensus sequence can be obtained from the sequencing reads clustered based on the sequences of the synthetic transposons and/or the duplicated gap sequences.

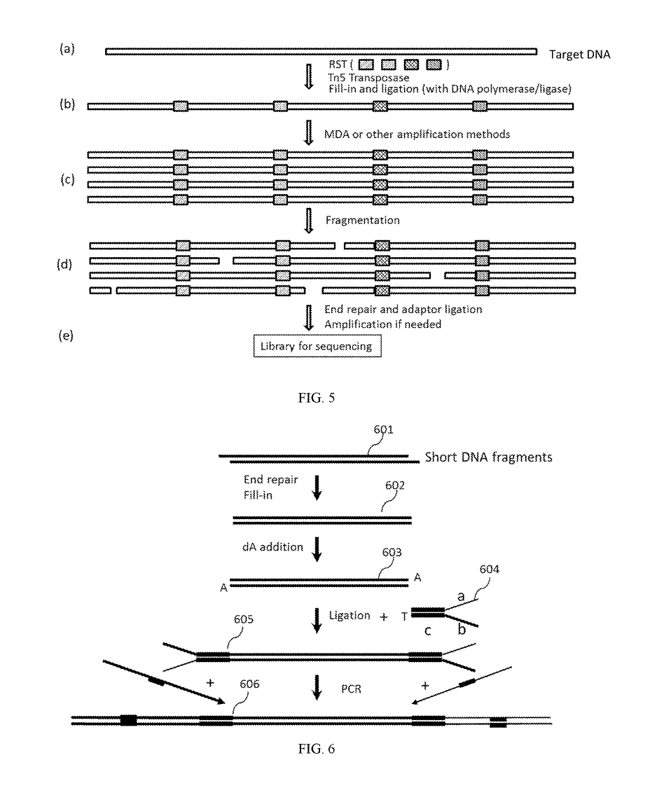

FIG. 7 shows an exemplary method for correcting errors (marked as “X”) or bias in sequencing reads by clustering short reads of template nucleic acids using molecular barcodes in Tn5 synthetic transposons on both ends of the template nucleic acids (e.g., prepared by strand displacement method in FIG. 4). In this example, for each sequencing sample, sequencing reads with identical (e.g., with no more than 1-base difference or similar setting if needed) mBCs on both ends of the reads are clustered together. With a minimum of 3 reads per identical mBC set, any error found in about 34% or less of sequencing reads in the set are corrected by taking on the identity of the majority base, resulting in a consensus sequence for the single template nucleic acid. Amplification or capture bias are removed as all sequencing reads having the same mBC pair is counted as a single copy of template molecule. Subsequently, the consensus sequences of the single template molecules are ‘stitched” together to provide a long phased sequence with haplotype information.

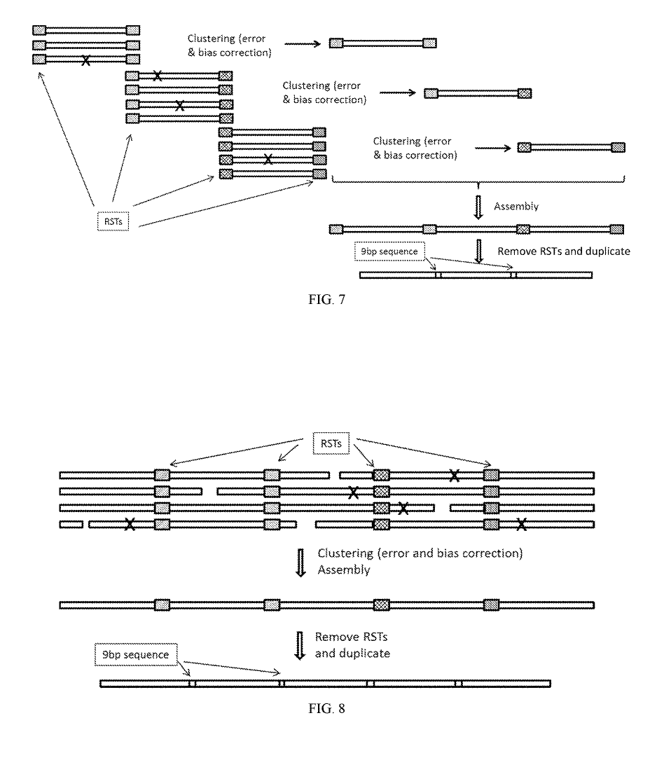

FIG. 8 shows an exemplary method for analyzing sequencing reads from libraries prepared using non-strand displacement methods (e.g., prepared using the method of FIG. 5). In such embodiments, a more intensive clustering of all sequencing reads can be done by aligning sequencing reads with perfectly or near-perfectly matched mBCs. Errors (marked as “X”) or bias in amplification or sequencing can be corrected by using the consensus sequence derived from a minimal of 3 reads aligned via the molecular barcodes of the synthetic transposons, which are clustered to provide a contig corresponding to a single target nucleic acid molecule. The transposase recognition sites flanking the molecular barcodes serve as unique identifiers to pinpoint the location of insertion sites, which can be indexed and aligned to the next sequencing reads with the identical molecular barcode sequences. The clustering step can be done sequentially by starting from one read or in parallel and then merged together.

It is possible that some sequences between 2 mBCs have longer than expected length due to nonrandom transposition or Poisson distribution. Using multiple homogenous cells may minimize or eliminate this problem. Additionally, repeating the method with replicate samples may help.

### Applications

The methods of analyzing or sequencing a target nucleic acid as described above can be used in a variety of applications, including, but not limited to de novo sequencing, resequencing, structural variation detection, copy number measurement, methylation analysis, genetic linkage analysis for identification of genes involved in disease etiology.

In some embodiments, there is provided a method of haplotyping a target nucleic acid (such as genomic DNA, for example, a chromosome) comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids whereby the contiguous sequence provides haplotype information of the target nucleic acid. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of haplotyping a target nucleic acid (such as genomic DNA, for example, a chromosome) comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; and (e) assembling a contiguous sequence of the target nucleic acid from the sequencing reads based on the molecular barcodes of the synthetic transposons in the template nucleic acids whereby the contiguous sequence provides haplotype information of the target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of haplotyping a target nucleic acid (such as genomic DNA, for example, a chromosome) comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids whereby the contiguous sequence provides haplotype information of the target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of assembly (such as de novo assembly or resequencing) of a target nucleic acid (such as genomic DNA, mitochondrial DNA, or microbial DNA), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids. In some embodiments, the method determines sequences of the target nucleic acids at single cell level. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of assembly (such as de novo assembly or resequencing) of a target nucleic acid (such as genomic DNA, mitochondrial DNA, or microbial DNA), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; and (e) assembling a contiguous sequence of the target nucleic acid from the sequencing reads based on the molecular barcodes of the synthetic transposons in the template nucleic acids. In some embodiments, the method determines sequences of the target nucleic acids at single cell level. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of assembly (such as de novo assembly or resequencing) of a target nucleic acid (such as genomic DNA, mitochondrial DNA, or microbial DNA), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; and (f) assembling a contiguous sequence of the target nucleic acid from the sequencing reads based on the molecular barcodes of the synthetic transposons in the template nucleic acids. In some embodiments, the method determines sequences of the target nucleic acids at single cell level. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

The methods of assembly disclosed herein may be used to generate reference genome sequences for human or other species or interest using multiple platforms or replicates with extreme low error rates (e.g., with lower than about 1/10, 1/100, 1/1000, or 1/10,000 the error rate of current reference genome sequences). The reference genomes can then be used to speed up the assembly process for new sequences from individuals in a species.

In some embodiments, there is provided a method of sequencing repetitive regions in a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; and (f) assembling a contiguous sequence covering the repetitive regions of the target nucleic acid from the sequencing reads based on the molecular barcodes of the synthetic transposons in the template nucleic acids. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of sequencing repetitive regions in a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; and (e) assembling a contiguous sequence covering the repetitive regions of the target nucleic acid from the sequencing reads based on the molecular barcodes of the synthetic transposons in the template nucleic acids. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence. In some embodiments, there is provided a method of sequencing repetitive regions in a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; and (f) assembling a contiguous sequence covering the repetitive regions of the target nucleic acid from the sequencing reads based on the molecular barcodes of the synthetic transposons in the template nucleic acids. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a mutation (such as SNP, indel, structural variation, translocation, or copy number variation) in a target nucleic acid (e.g., at single-cell level), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; and (g) comparing the contiguous sequence with a reference sequence to detect the mutation in the target nucleic acid. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a mutation (such as SNP, indel, structural variation, translocation, or copy number variation) in a target nucleic acid (e.g., at single-cell level), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; (e) assembling a contiguous sequence of the target nucleic acid from the sequencing reads based on the molecular barcodes of the synthetic transposons in the template nucleic acids; and (f) comparing the contiguous sequence with a reference sequence to detect the mutation in the target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a mutation (such as SNP, indel, structural variation, translocation, or copy number variation) in a target nucleic acid (e.g., at single-cell level), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; and (g) comparing the contiguous sequence with a reference sequence to detect the mutation in the target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a structural variation in a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; and (g) comparing the contiguous sequence with a reference sequence to detect the structural variation in the target nucleic acid. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a structural variation in a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; (e) assembling a contiguous sequence of the target nucleic acid from the sequencing reads based on the molecular barcodes of the synthetic transposons in the template nucleic acids; and (f) comparing the contiguous sequence with a reference sequence to detect the structural variation in the target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a structural variation in a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; and (g) comparing the contiguous sequence with a reference sequence to detect the structural variation in the target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of detecting a copy number variation in a target nucleic acid (such as a chromosome, exosome, or target sequences), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; (g) counting one copy of the target nucleic acid for all sequencing reads assembled to the contiguous sequence; and (h) comparing the copy number of the target nucleic acid with a reference to detect the copy number variation in the target nucleic acid. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the method further comprises capturing or enhancing the target nucleic acid or barcoded target nucleic acid, such as by using probes that hybridize to the target nucleic acid or barcoded target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence.

In some embodiments, there is provided a method of detecting a copy number variation in a target nucleic acid (such as a chromosome, exosome, or target sequences), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; (e) assembling a contiguous sequence of the target nucleic acid from the sequencing reads based on the molecular barcodes of the synthetic transposons in the template nucleic acids; (f) counting one copy of the target nucleic acid for all sequencing reads assembled to the contiguous sequence; and (g) comparing the copy number of the target nucleic acid with a reference to detect the copy number variation in the target nucleic acid. In some embodiments, the method further comprises capturing or enhancing the target nucleic acid or barcoded target nucleic acid, such as by using probes that hybridize to the target nucleic acid or barcoded target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence.

In some embodiments, there is provided a method of detecting a copy number variation in a target nucleic acid (such as a chromosome, exosome, or target sequences), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; (g) counting one copy of the target nucleic acid for all sequencing reads assembled to the contiguous sequence; and (h) comparing the copy number of the target nucleic acid with a reference to detect the copy number variation in the target nucleic acid. In some embodiments, the method further comprises capturing or enhancing the target nucleic acid or barcoded target nucleic acid, such as by using probes that hybridize to the target nucleic acid or barcoded target nucleic acid. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence.

Methods of bisulfite sequencing for analyzing methylation status of target nucleic acids (such as genomic DNA) are provided herein. DNA methylation is a widespread epigenetic modification that plays a pivotal role in the regulation of the genomes of diverse organisms. The most prevalent and widely studied form of DNA methylation in mammalian genomes occurs at the 5 carbon position of cytosine residues, usually in the context of the CpG dinucleotide. Microarrays, and more recently massively parallel sequencing, have enabled the interrogation of cytosine methylation (5 mC) on a genome-wide scale (Zilberman and Henikoff 2007). Methods of whole genome bisulfite sequencing that can be used to detect 5mC have been described (e.g., Cokus et al. 2008; Lister et al. 2009; Harris et al. 2010). Treatment of genomic DNA with sodium bisulfite chemically deaminates cytosines much more rapidly than 5mC, preferentially converting them to uracils (Clark et al. 1994). With massively parallel sequencing, these can be detected on a genome-wide scale at single base-pair resolution. Any of the known whole genome bisulfite sequencing workflows can be applied to genomic DNA samples barcoded with the synthetic transposons of the present application to provide methods of methylation analysis with high accuracy and efficiency.

In some embodiments, there is provided a method of analyzing methylation status of a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) subjecting the repaired barcoded target nucleic acid to bisulfite treatment; (d) amplifying the bisulfite-treated repaired barcoded target nucleic acid to provide a plurality of amplified barcoded target nucleic acids; and (e) fragmenting the plurality of amplified barcoded target nucleic acids thereby providing a library of template nucleic acids; (f) sequencing the library of template nucleic acids to obtain sequencing reads; (g) assembling a contiguous sequence of the target nucleic acid from the sequencing reads based on the molecular barcodes of the synthetic transposons in the template nucleic acids; and (h) comparing the contiguous sequence with a reference sequence of the target nucleic acids to determine methylation positions in the target nucleic acid. In some embodiments, the first transposase recognition site and the second transposase recognition site comprise 5-methyl dC. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing methylation status of a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) subjecting the library of template nucleic acids to bisulfite treatment; (e) sequencing the library of bisulfite treated template nucleic acids to obtain sequencing reads; (f) assembling a contiguous sequence of the target nucleic acid from the sequencing reads based on the molecular barcodes of the synthetic transposons in the template nucleic acids; and (g) comparing the contiguous sequence with a reference sequence of the target nucleic acids to determine methylation positions in the target nucleic acid. In some embodiments, the first transposase recognition site and the second transposase recognition site comprise 5-methyl dC. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing methylation status of a target nucleic acid (such as genomic DNA, for example, a chromosome), comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) subjecting the library of template nucleic acids to bisulfite treatment; (f) sequencing the library of bi-sulfite treated template nucleic acids to obtain sequencing reads; (g) assembling a contiguous sequence of the target nucleic acid from the sequencing reads based on the molecular barcodes of the synthetic transposons in the template nucleic acids; and (h) comparing the contiguous sequence with a reference sequence of the target nucleic acids to determine methylation positions in the target nucleic acid. In some embodiments, the first transposase recognition site and the second transposase recognition site comprise 5-methyl dC. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

Methods of determining chromosomal conformations (such a native 3-D structure of the genome) and protein-target nucleic acid interactions are provided herein. Various chromosome conformation capture techniques (see, for example, Barutcus A R et al, J. Cell Physiol, 231:31-35, 2016), such as 3C, circularized 3C (i.e., 4C), carbon-copy 3C (i.e., 5C), or chromatin immunoprecipitation-based methods (such as ChIP-loop), and genome conformation capture techniques may be combined with any one of the methods of inserting synthetic transposons described herein to assess chromosome interactions. Various chromatin immunoprecipitation (ChIP) methods (see, for example, P. Collas, Molecular Biotechnology 45(1):87-100, 2010) can be used to isolate protein-DNA complexes (such as chromatin-DNA complexes), which can then be barcoded with the synthetic transposons of the present application, and sequenced to determine the location in the genome that the protein (such as histones) are associated with.

In some embodiments, there is provided a method of analyzing conformation of a chromosome, comprising: (a) crosslinking the chromosome in vivo (such as within a cell); (b) isolating the crosslinked chromosome; (c) fragmenting (such as mechanically or enzymatically) the crosslinked chromosome to provide crosslinked chromosomal fragments; (d) ligating the ends of the crosslinked chromosomal fragments to provide ligated fragments; (e) reversing the ligated fragments to provide target nucleic acids; (f) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (g) contacting the barcoded target nucleic acids with a polymerase without strand displacement activity (such as T4 DNA polymerase), nucleotides (dNTPs), and a ligase to provide repaired barcoded target nucleic acids; (h) amplifying the repaired barcoded target nucleic acids to provide a plurality of amplified barcoded target nucleic acids; (i) fragmenting the plurality of amplified barcoded target nucleic acids thereby providing a library of template nucleic acids; (j) sequencing the library of template nucleic acids to obtain sequencing reads; (k) assembling contiguous sequences of the target nucleic acid from the sequencing reads based on the molecular barcodes of the synthetic transposons in the template nucleic acids; and (1) comparing the contiguous sequences with a reference sequence of the chromosome to determine conformation of the chromosome. In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing conformation of a chromosome, comprising: (a) crosslinking the chromosome in vivo (such as within a cell); (b) isolating the crosslinked chromosome; (c) fragmenting (such as mechanically or enzymatically) the crosslinked chromosome to provide crosslinked chromosomal fragments; (d) ligating the ends of the crosslinked chromosomal fragments to provide ligated fragments; (e) reversing the ligated fragments to provide target nucleic acids; (f) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (g) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (h) amplifying the fragments to provide a library of template nucleic acids; (i) sequencing the library of template nucleic acids to obtain sequencing reads; (j) assembling contiguous sequences of the target nucleic acids from the sequencing reads based on the molecular barcodes of the synthetic transposons in the template nucleic acids; and (k) comparing the contiguous sequences with a reference sequence of the chromosome to determine conformation of the chromosome. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

In some embodiments, there is provided a method of analyzing conformation of a chromosome, comprising: (a) crosslinking the chromosome in vivo (such as within a cell); (b) isolating the crosslinked chromosome; (c) fragmenting (such as mechanically or enzymatically) the crosslinked chromosome to provide crosslinked chromosomal fragments; (d) ligating the ends of the crosslinked chromosomal fragments to provide ligated fragments; (e) reversing the ligated fragments to provide target nucleic acids; (f) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (g) contacting the barcoded target nucleic acid with a polymerase without strand-displacement activity (such as T4 DNA polymerase), and nucleotides (dNTPs), and a ligase to provide a repaired barcoded target nucleic acid; (h) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (i) amplifying the fragments to provide a library of template nucleic acids; (j) sequencing the library of template nucleic acids to obtain sequencing reads; (k) assembling contiguous sequences of the target nucleic acids from the sequencing reads based on the molecular barcodes of the synthetic transposons in the template nucleic acids; and (l) comparing the contiguous sequences with a reference sequence of the chromosome to determine conformation of the chromosome. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence. In some embodiments, the method further comprises counting one copy of the target nucleic acid for all sequencing reads assembled to the contiguous sequence.

Any of the methods and applications described above can be used for diagnosing a disease or a condition in an individual based on the sequence, contiguity information (such as haplotype or 3-dimensional chromosome conformation), and/or quantity of a target nucleic acid in the individual. The target nucleic acid may be present in a sample obtained from the individual, including, but not limited to, biopsy sample, buccal swap, blood sample, or sample of other bodily fluid. In some embodiments, the target nucleic acid of the individual is compared to a reference from a healthy individual to provide the diagnosis.

In some embodiments, there is provided a method of diagnosing a disease or a condition of an individual based on status of a target nucleic acid (such as genomic DNA, for example, a chromosome) from the individual, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode (such as partially single-stranded, single-stranded or double-stranded) disposed between the first transposase recognition site and the second transposase recognition site, and wherein each synthetic transposon comprises a different molecular barcode; (b) contacting the barcoded target nucleic acid with a polymerase without strand displacement activity (such as T4 DNA polymerase), nucleotides (dNTPs), and a ligase to provide a repaired barcoded target nucleic acid; (c) amplifying the repaired barcoded target nucleic acid to provide a plurality of amplified barcoded target nucleic acids; (d) fragmenting the plurality of amplified barcoded target nucleic acids thereby providing a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; (g) optionally counting one copy of the target nucleic acid for all sequencing reads assembled to the contiguous sequence; and (h) providing a diagnosis based on the contiguous sequence and/or the copy number of the target nucleic acid. In some embodiments, the diagnosis comprises mutations, such as structural variations or copy number variations in a diseased tissue (such as tumor). In some embodiments, the molecular barcode is double-stranded. In some embodiments, the molecular barcode comprises a single-stranded region. In some embodiments, each synthetic transposon comprises one or two terminal hairpin structures. In some embodiments, each synthetic transposon comprises two double-stranded ends with no terminal hairpin structures. In some embodiments, the 5′ termini of the two double-stranded ends are phosphorylated. In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence.

In some embodiments, there is provided a method of diagnosing a disease or a condition of an individual based on status of a target nucleic acid (such as genomic DNA, for example, a chromosome) from the individual, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a double-stranded molecular barcode disposed between the first transposase recognition site and the second transposase recognition site, and wherein each synthetic transposon comprises a different molecular barcode; (b) contacting the barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (c) amplifying the fragments to provide a library of template nucleic acids; (d) sequencing the library of template nucleic acids to obtain sequencing reads; (e) assembling a contiguous sequence of the target nucleic acid from the sequencing reads based on the molecular barcodes of the synthetic transposons in the template nucleic acids; (f) optionally counting one copy of the target nucleic acid for all sequencing reads assembled to the contiguous sequence; and (g) providing a diagnosis based on the contiguous sequence and/or the copy number of the target nucleic acid. In some embodiments, the diagnosis comprises mutations, such as structural variations or copy number variations in a diseased tissue (such as tumor). In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence.

In some embodiments, there is provided a method of diagnosing a disease or a condition of an individual based on status of a target nucleic acid (such as genomic DNA, for example, a chromosome) from the individual, comprising: (a) contacting the target nucleic acid with a composition comprising a plurality of synthetic transposons and a transposase (such as Tn5 transposase, e.g., hyperactive Tn5 transposase) under a condition that allows insertion of at least a portion of the plurality of synthetic transposons into the target nucleic acid to provide a barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) contacting the barcoded target nucleic acid with a polymerase without strand-displacement activity (such as T4 DNA polymerase), and nucleotides (dNTPs), and a ligase to provide a repaired barcoded target nucleic acid; (c) contacting the repaired barcoded target nucleic acid with a polymerase with strand displacement activity (such as Klenow fragment without 3′-5′ exonuclease activity) and nucleotides (such as dNTPs) to provide fragments of the repaired barcoded target nucleic acid, wherein each fragment comprises a synthetic transposon at one end; (d) amplifying the fragments to provide a library of template nucleic acids; (e) sequencing the library of template nucleic acids to obtain sequencing reads; (f) assembling a contiguous sequence of the target nucleic acid from the sequencing reads based on the molecular barcodes of the synthetic transposons in the template nucleic acids; (g) optionally counting one copy of the target nucleic acid for all sequencing reads assembled to the contiguous sequence; and (h) providing a diagnosis based on the contiguous sequence and/or the copy number of the target nucleic acid. In some embodiments, the diagnosis comprises mutations, such as structural variations or copy number variations in a diseased tissue (such as tumor). In some embodiments, the sequencing reads are assembled to provide the contiguous sequence of the target nucleic acid by steps comprising: (i) identifying sequences of the synthetic transposons in the sequencing reads; (ii) aligning sequencing reads having the same molecular barcodes in the synthetic transposons to provide aligned sequencing reads; and (iii) clustering the aligned sequencing reads based on the molecular barcodes in the synthetic transposons to provide the contiguous sequence of the target nucleic acid. In some embodiments, wherein each synthetic transposon inserted in the target nucleic acid is flanked by a pair of single-stranded gaps having duplicated sequences endogenous to the target nucleic acid, the duplicated sequences are further used to assemble the contiguous sequence.

Some embodiments described herein comprise comparing the contiguous sequence of the target nucleic acid in a sample to a reference sequence, the copy number of the target nucleic acid in a sample to a reference value, and/or comparing the contiguous sequence and/or copy number of the target nucleic acid of one sample to that of a reference sample. The reference sequence and reference values may be obtained from a database. The reference sample may be a sample from a healthy or wildtype individual, tissue, or cell. For example, in some embodiments, the target nucleic acid from a tumor cell of an individual is analyzed and compared to the nucleic acid from a healthy cell of the same individual to provide a diagnosis.

Examples of applications are described below, as well as in the “Examples” section. For example, using methods of analyzing a target nucleic acid described in the “Methods of analysis” section, once the sequencing reads are constructed back to a single target molecule level, whether the DNA is obtained from homogenous cells can be deduced, as it is expected that for a certain chromosome in a diploid organisms, some sequencing reads would be mapped to one copy of the chromosome, while the other sequencing reads would be mapped to the second copy of the chromosome in a sample from homogenous cells. At the single cell level for normal cells, sequencing reads are expected to map to two original target molecules for a chromosome, each belonging to one of the two chromosomes (one paternal and one maternal). If multiple cells are used, molecules can be clustered into paternal and maternal chromosomes. Chromosome number, copy number or structural changes can thus be detected. The number of cells used for the assay depends on the purpose of the assay. In most cases for high quality clinical sequencing, 10-50 cells might be sufficient. Sequencing of a higher number of cells requires a larger number of sequencing reads to detect variations, such as mutations. Although amplification bias can be removed, plenty of read coverage (for example, at least 3) needs to be obtained. A sufficient read coverage may be especially important for sequencing high G/C or A/T-rich or repetitive regions. Insertion of synthetic transposons into such regions with a balanced G/C percentage could facilitate sequencing of these regions.

Although human individuals are 99.5% similar in the genomes, each individual has about 10 million single nucleotide polymorphisms (SNPs), private alleles or structural changes. Small somatic mutations, such as substitution, insertion, deletion or large structural changes (e.g., translocation or multiplication) could accumulate over time, leading to tumor formation or changes in cells. Epigenetic modification such as methylation is abundant and quite different among different cells. Therefore, it is interesting to understand such changes at single cell level. As single molecules are detected by embodiments of methods described herein, the methods can be used to detect sequence changes such as mutations in these cells accurately. For example, targeted amplification or exome capture can be used to enrich the desired templates, allowing specific targets to be sequenced. Moreover, there are hundreds of copies of mitochondria present per cell, but each mitochondrion has slightly different sequences. Embodiments of methods described herein allow sequencing of all individual mitochondria at single molecule level. On the other hand, millions of different microbes are living with each human individual and identification of each microbe at single cell level is also important, especially considering the similarity among multiple different species of microbes.

Theoretically, with the methods described herein, long target nucleic acids are preferred to obtain uninterrupted haplotype information with unequivocal sequences. By contrast, long target nucleic acids may not be well resolved with methods involving diluting single molecules to single compartments (such as wells) and tagging samples within the same compartment with the same sample tag. For example, with the dilution method, repetitive sequences in long target nucleic acids may not be aligned unequivocally.

### Kits and Articles of Manufacture

The present application further provides kits and articles of manufacture comprising a plurality of any of the synthetic transposons described herein, and for methods of library preparation, analyzing target nucleic acids, or various applications described herein.

In some embodiments, there is provided a kit for preparing a library of template nucleic acids, comprising: (a) a plurality of synthetic transposons each comprising a first transposase recognition site, a second transposase recognition site, and a molecular barcode disposed between the first transposase recognition site and the second transposase recognition site, wherein each synthetic transposon comprises a different molecular barcode, and wherein the molecular barcode comprises a single-stranded region; (b) a transposase that recognizes the first transposon recognition site and the second transposon recognition site; and (c) instructions for preparing the library of template nucleic acids. In some embodiments, the kit further comprises a polymerase without strand displacement activity, such as T4 DNA polymerase. In some embodiments, the kit further comprises a ligase. In some embodiments, the kit further comprises nucleotides (such as dNTPs). In some embodiments, the kit further comprises a polymerase with strand displacement activity (such as a Klenow fragment without 3′-5′ exonuclease activity). In some embodiments, the transposase is Tn5 transposase, including a modified Tn5 transposase with enhanced activity, such as EZ-Tn5™.

In some embodiments, there is provided a kit for preparing a library of template nucleic acids, comprising: (a) a plurality of synthetic transposons each comprising a first transposase recognition site, a second transposase recognition site, and a molecular barcode disposed between the first transposase recognition site and the second transposase recognition site, wherein each synthetic transposon comprises a different molecular barcode, wherein the molecular barcode comprises a single-stranded region, wherein each synthetic transposon comprises two double-stranded ends with no terminal hairpin structures, wherein the 5′ termini of the two double-stranded ends are unphosphorylated, and wherein the 5′ terminus adjacent to the single-stranded region is phosphorylated; (b) a transposase that recognizes the first transposon recognition site and the second transposon recognition site; and (c) instructions for preparing the library of template nucleic acids. In some embodiments, the kit further comprises a polymerase without strand displacement activity, such as T4 DNA polymerase, and a polymerase with strand displacement activity (such as a Klenow fragment without 3′-5′ exonuclease activity). In some embodiments, the kit further comprises a ligase. In some embodiments, the kit further comprises nucleotides (such as dNTPs). In some embodiments, the transposase is Tn5 transposase, including a modified Tn5 transposase with enhanced activity, such as EZ-Tn5™.

In some embodiments, there is provided a kit for preparing a library of template nucleic acids, comprising: (a) a plurality of synthetic transposons each comprising a first transposase recognition site, a second transposase recognition site, and a double-stranded molecular barcode disposed between the first transposase recognition site and the second transposase recognition site, and wherein each synthetic transposon comprises a different molecular barcode; (b) a transposase that recognizes the first transposon recognition site and the second transposon recognition site; and (c) instructions for preparing the library of template nucleic acids. In some embodiments, the kit further comprises a polymerase. In some embodiments, the kit further comprises nucleotides (such as dNTPs). In some embodiments, the polymerase is a DNA polymerase with strand displacement activity, such as a Klenow fragment without 3′-5′ exonuclease activity. In some embodiments, the polymerase is a DNA polymerase without strand displacement activity, such as T4 DNA polymerase. In some embodiments, the kit further comprises a ligase. In some embodiments, the transposase is Tn5 transposase, including a modified Tn5 transposase with enhanced activity, such as EZ-Tn5™.

In some embodiments, there is provided a kit for preparing transposition comprising: (a) a transposase; (b) Random synthetic transposon (RST) recognized by the transposase; (c) DNA polymerase for filling-in gaps; (d) Buffer with dNTPs, salts and cofactors; and (e) optionally ligase for nick ligation. In some embodiments, said transposase is a modified Tn5 with enhanced activity or similar one. In some embodiments, said DNA polymerase could be T4 DNA polymerase for fill-in only or Klenow Fragment without 3′-5′ exonuclease activity for fill-in and displacement.

The kits may contain one or more additional components, such as containers, buffers, reagents, cofactors, or additional agents, such as agents for isolating high molecular weight nucleic acids (such as chromosomes) from cells. The kit components may be packaged together and the package may contain or be accompanied by instructions for using the kit.

It will be appreciated by persons skilled in the art the numerous variations, combinations and/or modifications may be made to the invention as shown without departing from the spirit of the inventions as broadly described.

### EXAMPLES

The examples below are intended to be purely exemplary of the invention and should therefore not be considered to limit the invention in any way. The following examples and detailed description are offered by way of illustration and not by way of limitation.

### Example 1: Whole Genome Sequencing of Genomic DNA from a Human Individual to Use as a Reference Genome

An exemplary method of preparing a sequencing library for whole genome sequencing of a genomic DNA sample from a human individual is described below.

Human gDNA is extracted from a buccal swap or a drop of blood, and the purity and yield of the gDNA is measured. A composition comprising a plurality of synthetic transposons each having two 19-bp Tn5 recognition sites flanking a molecular barcode comprising 20 randomly designed nucleotides (N), fixed bases, and other degenerately designed bases as shown in FIG. 2A is prepared. Duplicate samples of the gDNA inserted with the plurality of synthetic transposons are prepared. In each sample, about 0.3 ng gDNA is used to contact with the composition comprising the plurality of synthetic transposons under a condition that allows insertion at a frequency of about 150-bp between adjacent transposition sites. The 9 nt single-stranded gaps are filled-in with dNTPs and DNA polymerase without strand displacement activity, such as T4 DNA polymerase. The nicks are ligated with E coli ligase, and the ligation step can be done together with the gap fill-in step. Qiagen's Replig-g kit is used to do whole genome amplification. The amplified products are sheared with physical (e.g., Covaris's DNA shearing equipment) or enzymatic (e.g., DNase I) fragmentation methods to an average length of about 500-bp. Fragments with the desired length (˜500-bp) are purified with AMPure XP beads. NEBnext DNA library Prep reagent sets for Illumina are used to prepare a library from the purified fragments for sequencing, including steps of end repair and 5′ phosphorylation, dA-tailing, adaptor ligation with NEBnext adaptors, UDG treatment, PCR with sample tags and common primers. The PCR products are pooled, purified, and quantified to provide the sequencing library, which is sequenced with a pair-end sequencing technique (2×300 bases) on an Illumina instrument. The sequence signature at each insertion site, including 9-bp sequence+ME1+mBC sequence+ME2+9-bp duplicate, is used in data analysis to obtain the assembled human genome with high quality with haplotype information with any structural and base changes. It is noted that in the future, fragment sizes of 750 bp can be used on pair-ended sequencing platforms having 2×500 base read length.

### Example 2: Targeted Capture for Copy Number Change in Tumor Cells

An exemplary method of detecting copy number variations in tumor cells is described below.

Human gDNA samples are extracted from both tumor tissues and surrounding normal tissues for comparison. The purity and yield of the samples are measured. Typically, gDNA in the range of ng (e.g., for normal or tumor tissues) to μg (e.g., for tumor tissues usually) is used per experiment. A high amount of tumor tissues is useful for identifying rare and secondary changes, albeit yielding more sequence reads. A composition comprising a plurality of synthetic transposons each having two 19-bp Tn5 recognition sites flanking a molecular barcode comprising 20 randomly designed nucleotides (N), fixed bases, and other degenerately designed bases as shown in FIG. 2A is prepared. Duplicate samples of the gDNA inserted with the plurality of synthetic transposons are prepared. In each sample, gDNA (for example, about 3 ng) is used to contact with the composition comprising the plurality of synthetic transposons under a condition that allows insertion at a frequency of at least 500-bp (for example, about 150-bp) between adjacent transposition sites for both tumor and normal samples. The single-stranded gaps are filled-in with dNTPs and a DNA polymerase with strand displacement activity such as Klenow fragment (3′-5′Exo-) to provide fragments of target nucleic acids, having a synthetic transposon sequence at each end. A NEBnext DNA library prep kit for Illumina is used to add adaptors to the fragments, and amplified by to add the sample tags and common primers. Exome capture probes from vendors or custom-designed probes are used to capture the desired sequences. As each sample is tagged with a specific sample tag, it's possible to pool the samples before capture. The captured product is optionally purified, and quantified. The resulting sequencing library is sequenced with a pair-end sequencing technique (2×300 bases) on an Illumina instrument. In data analysis, two fragments having matching ends, i.e., one with “A”+ME1+mBC sequence+ME2+9-nt, and the other fragment with “A”+reverse complementary of (ME2+mBC sequence+ME1+9-nt), can be linked together as these fragments represent contiguous sequences prior to transposition events. The exome or targeted sequences are assembled based on the synthetic transposons, and copy number changes of the targeted regions are determined. In this example, it is not necessary to sequence the amplicons completely as counting of the target sequences is the main focus, and the synthetic transposons allow mapping of the redundant specific sequencing reads to single target molecules.

1. A composition comprising a plurality of synthetic transposons, each synthetic transposon comprising a first transposase recognition site, a second transposase recognition site, and a molecular barcode disposed between the first transposase recognition site and the second transposase recognition site, wherein each synthetic transposon comprises a different molecular barcode.

2. The composition of claim 1, wherein the molecular barcode is double-stranded.

3. The composition of claim 1, wherein the molecular barcode comprises a single-stranded region.

4. The composition of claim 1, wherein the molecular barcode comprises at least about 5 randomly or degenerately designed nucleotides.

5-10. (canceled)

11. The composition of claim 3, wherein the 5′ terminus adjacent to the single-stranded region is phosphorylated.

12. A method of preparing a library of template nucleic acids, comprising:
(a) contacting a target nucleic acid with the composition of claim 1, and a transposase under a condition that allows insertion of at least a portion of the plurality of synthetic transposons into the target nucleic acid to provide a barcoded target nucleic acid;
(b) contacting the barcoded target nucleic acid with a polymerase without strand displacement activity, nucleotides, and a ligase to provide a repaired barcoded target nucleic acid;
(c) amplifying the repaired barcoded target nucleic acid to provide a plurality of amplified barcoded target nucleic acids; and
(d) fragmenting the plurality of amplified barcoded target nucleic acids thereby providing the library of template nucleic acids.

13. A method of preparing a library of template nucleic acids, comprising:
(a) contacting a target nucleic acid with the composition of claim 11, and a transposase under a condition that allows insertion of at least a portion of the plurality of synthetic transposons into the target nucleic acid to provide a barcoded target nucleic acid;
(b) contacting the barcoded target nucleic acid with a polymerase without strand displacement activity, nucleotides, and a ligase to provide a repaired barcoded target nucleic acid;
(c) contacting the repaired barcoded target nucleic acid with a polymerase with strand displacement activity and nucleotides to provide fragments of the repaired barcoded target nucleic acid, wherein each fragment comprises a synthetic transposon at one end; and
(d) amplifying the fragments to provide the library of template nucleic acids.

14. (canceled)

15. A method of preparing a library of template nucleic acids, comprising:
(a) contacting a target nucleic acid with the composition of claim 1, and a transposase under a condition that allows insertion of at least a portion of the plurality of synthetic transposons into the target nucleic acid to provide a barcoded target nucleic acid;
(b) contacting the barcoded target nucleic acid with a polymerase with strand displacement activity and nucleotides to provide fragments of the repaired barcoded target nucleic acid, wherein each fragment comprises a synthetic transposon at one end; and
(c) amplifying the fragments to provide the library of template nucleic acids.

16. (canceled)

17. The method of claim 1, wherein the target nucleic acid is contacted with the plurality of synthetic transposons and the transposase in vitro.

18. The method of claim 17, wherein the plurality of synthetic transposons and the transposase are pre-mixed prior to contacting the target nucleic acid.

19. The method of claim 12, wherein the target nucleic acid is contacted with the plurality of synthetic transposons and the transposase in vivo.

20. (canceled)

21. The method of claim 12, wherein the target nucleic acid is selected from the group consisting of cDNA, genomic DNA, bisulfite-treated DNA, and crosslinked DNA.

22. The method of claim 12, wherein the plurality of synthetic transposons are inserted into the target nucleic acid at a frequency of at least once per about 500 bases.

23-24. (canceled)

25. A method of analyzing a target nucleic acid, comprising:
(a) preparing a library of template nucleic acids from the target nucleic acid using the method of claim 12;
(b) sequencing the library of template nucleic acids to obtain sequencing reads; and
(c) assembling a contiguous sequence of the target nucleic acid from the sequencing reads based on the molecular barcodes of the synthetic transposons in the template nucleic acids.

26-27. (canceled)

28. The method of claim 25, wherein each synthetic transposon inserted in the target nucleic acid is flanked by a pair of single-stranded gaps having duplicated sequences endogenous to the target nucleic acid, and wherein the duplicated sequences are further used to assemble the contiguous sequence.

29. The method of claim 25, further comprising counting one copy of the target nucleic acid for all sequencing reads assembled to the contiguous sequence.

30. The method of claim 25, wherein the method is used for genome assembly, haplotyping, detection of mutation, chromosomal conformation analysis, or methylation analysis.

31. (canceled)

32. A barcoded target nucleic acid comprising a plurality of synthetic transposons inserted randomly or substantially randomly among the endogenous sequence of the barcoded target nucleic acid, wherein each synthetic transposon comprises a first transposase recognition site, a second transposase recognition site, and a molecular barcode disposed between the first transposase recognition site and the second transposase recognition site, wherein each synthetic transposon comprises a different molecular barcode.

33-34. (canceled)

35. A cell comprising the barcoded target nucleic acid of claim 32.

36. A kit for preparing a library of template nucleic acids, comprising:
(a) the composition of claim 1;
(b) a transposase that recognizes the first transposon recognition site and the second transposon recognition site; and
(c) instructions for preparing the library of template nucleic acids.

37-42. (canceled)
